(19) United States
(12) Patent Application Publication
(10) Pub. No.: US 2025/0361227 A1
(43) Pub. Date: Nov. 27, 2025

(54) INHIBITORS OF FLI1 AND ERG

(71) Applicant: UNIVERSITY OF VIRGINIA PATENT FOUNDATION, Charlottesville, VA (US)

(72) Inventors: John H. BUSHWELLER, Crozet, VA (US); Venkata Sesha Kiran Kumar SRIMATH TIRUMALA, Charlottesville, VA (US); Adam Michael BOULTON, Medina, OH (US); Ashish KABRA, Distric Nagaur (raj) (IN)

(21) Appl. No.: 18/844,280

(22) PCT Filed: Mar. 13, 2023
(86) PCT No.: PCT/US2023/064237

§ 371 Date: Sep. 5, 2024

Related U.S. Application Data
(60) Provisional application No. 63/319,353, filed on Mar. 13, 2022.

(51) Int. Cl. C07D 413/14; A61K 31/423; A61P 35/00

(52) U.S. Cl. CPC C07D 413/14; A61K 31/423; A61P 35/00

(57) ABSTRACT

The invention relates to inhibitors of EWS-FLI1, pharmaceutical compositions containing the inhibitors, and methods of treating cancer, including Ewing sarcoma, leukemia, diffuse large B-cell lymphoma (DLBCL), and prostate cancer, comprising the administration of the inhibitors and pharmaceutical compositions thereof.

### CROSS REFERENCE TO RELATED APPLICATIONS

This application claims priority to U.S. Provisional Application No. 63/319,353, filed on Mar. 13, 2022, the disclosure of which is hereby incorporated by reference.

### STATEMENT OF U.S. GOVERNMENT SUPPORT

This invention was made with government support under CA231637 awarded by the National Institutes of Health. The U.S. government has certain rights in the invention.

### TECHNICAL FIELD

The invention relates to inhibitors of EWS-FLI1, pharmaceutical compositions containing the inhibitors, and methods of treating cancer, including Ewing sarcoma, leukemia, diffuse large B-cell lymphoma (DLBCL), and prostate cancer, comprising the administration of the inhibitors and pharmaceutical compositions thereof.

### BACKGROUND

Ewing sarcoma (EWS), the second most common pediatric tumor involving bone in children and young adults, remains an unmet clinical need. Five-drug combination chemotherapy, and the delivery of interval compressed chemotherapy cycles, have improved outcomes for patients with localized disease. For these children, event-free survival approaches 70%. Outcomes for children with metastatic disease, however, remain unchanged despite these intensifications of therapy, with only 30% achieving long-term disease control. Relapsed disease is nearly always fatal. Thus, new therapeutic approaches distinct from traditional cytotoxic chemotherapy are needed for these children, particularly those with metastatic or relapsed Ewing sarcoma.

Ewing sarcoma is directly linked to a chromosomal translocation event between the EWS gene and a member of the ETS transcription factor family, most frequently FLI1. The resulting fusion protein, EWS-FLI1, is the dominant driver of Ewing sarcoma development and is required for disease maintenance and progression. Fusions with the ETS family member ERG are also observed in a subset of patients. The (11;22)(q24;q12) translocation, which leads to expression of EWS-FLI1, is identified in 85% of Ewing sarcoma cases. This fusion oncoprotein encodes a transcription factor translocation in which the DNA-binding domain (DBD) of the ETS transcription factor FL11 is fused to the transactivation domain of the EWSR1 gene, leading to aberrant gene expression. The DNA binding capability of the fusion proteins is essential, making this a valid target for inhibitor development.

There are few examples of well-validated drug-like molecules that inhibit protein-DNA binding, likely due to the highly positively charged and convex nature of the DNA binding interface on DNA binding domains. To overcome this, the present invention targets the auto-inhibition of ERG and FLI1 to mediate inhibition of EWS-FLI1 and EWS-ERG. It was previously shown that ERG is auto-inhibited by regions of the protein flanking the DNA binding domain (17) and it was confirmed this is also the case for the highly homologous FLI1. Compounds were screened for those that selectively inhibit an auto-inhibited construct of ERG but not the isolated DNA binding domain (Ets domain). Optimized versions of these compounds demonstrate selectivity for ERG and FLI1 over other members of the Ets family of transcription factors, highlighting the potential for this approach to achieve selective inhibition.

High throughput screening failed to provide useful hits, so fragment screening was utilized and hits were identified that were verified by NMR to bind to ERG. Several fragments with IC₅₀ values of ˜1 mM were identified and medicinal chemistry approaches were used to improve the potency to the M range. Three classes of fragments, 9F1, 9B5 and 6H6 were pursued. 9F1 has the following chemical structure:

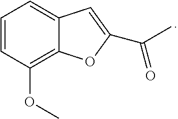

9B5 has the following chemical structure:

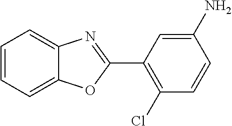 [SMILES: Nc1ccc(Cl)c(-c2nc3ccccc3o2)c1]

6H6 has the following chemical structure:

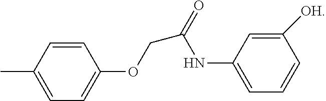 [SMILES: Cc1ccc(OCC(=O)Nc2cccc(O)c2)cc1]

86 analogs of 9F1, 120 analogs of 9B5, and 55 analogs of 6H6 were synthesized, with the most potent compounds identified being KK-16-69 (IC₅₀ 99 μM), KK-19-109 (IC₅₀ 63 μM) and KK-22-93 (IC₅₀ 88 μM), respectively. KK-16-69 has the following chemical structure:

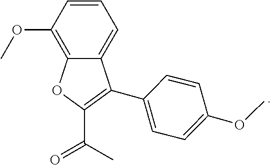

KK-19-109 has the following chemical structure:

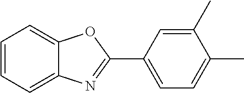 [SMILES: Cc1ccc(-c2nc3ccccc3o2)cc1C]

KK-22-93 has the following chemical structure:

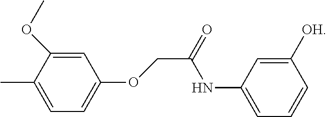 [SMILES: COc1cc(OCC(=O)Nc2cccc(O)c2)ccc1C]

These compounds clearly demonstrate selectivity for ERG and FL11 over other members of the Ets family of transcription factors, highlighting the potential for this approach to achieve selective inhibition of specific members of a family of transcription factors.

To achieve higher potency and longer duration of action of these inhibitors, approaches to convert them to covalent irreversible inhibitors were explored. To that end, a library of Cys reactive compounds were screened to identify covalent inhibitors of DNA binding. Three hits from this screen were elaborated upon and a series of analogs of one hit were evaluated for sites to link to. Hetero-bivalent compounds that link one of the autoinhibition fragment inhibitors to one of the covalent inhibitor derivatives were synthesized using polyethylene glycol-based linkers and click chemistry to link them together. Several derivatives with varying linker lengths were synthesized.

To assay such compounds time-dependent inhibition of ERG-DNA binding was recorded using a fluorescence polarization (FP) based assay and the rates were fitted to derive K_{I} and k_{inact} for the compounds which were ranked based on the k_{inact}/K_{I} values as has been described previously. These compounds clearly demonstrate the time-dependent inhibition characteristic of irreversible inhibitors.

This invention explores novel approaches to modulating the aberrant transcription driven by the EWS-FLI1(ERG) fusion proteins present in Ewing sarcoma tumors.

### SUMMARY OF THE INVENTION

The invention relates to a compound of formula (I):

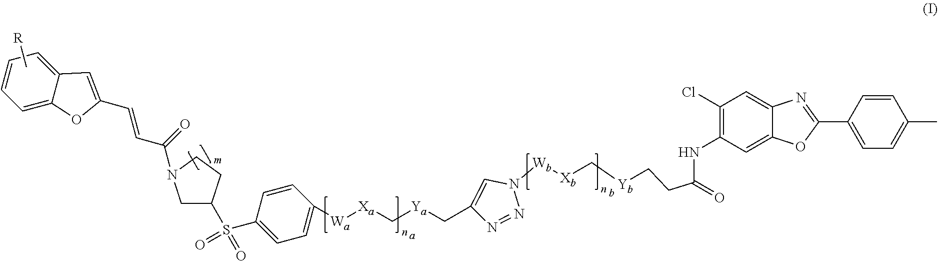

wherein:
R is, for each of the available binding sites, independently selected from the group consisting of H, F, Cl, Br, I, CH₃, OCH₃, CF₃, OCF₃, NO₂, NH₂, OH, N(CH₃)₂, CN, COCH₃, CONH₂, SO₂CH₃, and SO₂NH₂;
m is an integer from 1 to 4;
Wₐ, Xₐ, and Yₐ are for each occurrence independently O, CH₂, NH, cycloalkyl, benzyl, heterocycloalkyl, or heteroaryl, and one or more of Wₐ, Xₐ, and Yₐ are optionally not present, wherein the cycloalkyl, benzyl, heterocycloalkyl, and heteroaryl groups are independently unsubstituted or substituted, independently for each of the available binding sites, by H, F, Cl, Br, I, CH₃, OCH₃, CF₃, OCF₃, NO₂, NH₂, OH, N(CH₃)₂, CN, COCH₃, CONH₂, SO₂CH₃, or SO₂NH₂;
nₐ is an integer from 0 to 10;
W_{b}, X_{b}, and Y_{b} are for each occurrence independently O, CH₂, NH, cycloalkyl, benzyl, heterocycloalkyl, or heteroaryl, and one or more of W_{b}, X_{b}, and Y_{b} are optionally not present, wherein the cycloalkyl, benzyl, heterocycloalkyl, and heteroaryl groups are independently unsubstituted or substituted, independently for each of the available binding sites, by H, F, Cl, Br, I, CH₃, OCH₃, CF₃, OCF₃, NO₂, NH₂, OH, N(CH₃)₂, CN, COCH₃, CONH₂, SO₂CH₃, or SO₂NH₂; and
n_{b} is an integer from 0 to 10;
   or a pharmaceutically acceptable salt thereof.

The invention further relates to a pharmaceutical composition comprising a compound of formula (I) and a pharmaceutically acceptable excipient.

The invention further relates to methods of treating cancer, comprising administering to a subject in need thereof a therapeutically effective amount of a compound of formula (I) or a pharmaceutical composition of the invention.

The cancer being treated may be Ewing sarcoma, leukemia, diffuse large B-cell lymphoma (DLBCL), and/or prostate cancer.

### BRIEF DESCRIPTION OF THE FIGURES

FIG. 1 illustrates that ERG and FLI1 are members of the Ets transcription factor family.

FIG. 2 shows the EWS-FLI1 transcription factor schematically.

FIG. 3 shows the oncogenic fusion gene of EWSR1 and the Ets family member.

FIG. 4 shows the mechanisms of transcriptional regulation driven by EWS-FLI1.

FIG. 5 illustrates the principle of auto-inhibition.

FIG. 6 shows the primary sequence of the ERG protein and of additional constructs of the ERG protein created to analyze autoinhibition.

FIG. 7 shows isothermal titration calorimetry data for the binding of 3 of the constructs shown in FIG. 6 to DNA (A: ERG, B: ERGi, C: ERGu).

FIG. 8 shows the 3D structures of ERGu (A) and ERGi (B) solved using x-ray crystallography and a surface representation of the structure of ERGi (C).

FIG. 9 shows a representative plot of FP assays for the fragments 9B5 (black/squares) and KK-19-109 (red/circles) with auto-inhibited ERG.

FIG. 10 shows NMR chemical shift changes observed in an ¹⁵N-¹H HSQC NMR spectrum of ERGi alone and ERGi plus one of the active fragments.

FIG. 11 shows a representative plot of FP assays for the fragments 9F1 (black/squares) and KK-16-69 (red/circles) with auto-inhibited FLI1.

FIG. 12A shows compound KK-36-84 assayed using time-dependent ERG-DNA assay.

FIG. 12B shows compounds KK-36-25 and KK-36-105 assayed using the time-dependent ERG-DNA assay.

FIG. 12C shows compound KK-36-70 assayed using the time-dependent ERG-DNA assay.

FIG. 12D shows compound KK-36-111 assayed using the time-dependent ERG-DNA assay.

FIG. 13A shows the inhibition constants (K_{I}, k_{inact}) of irreversible inhibitor KK-36-84.

FIG. 13B shows the inhibition constants (K_{I}, k_{inact}) of irreversible inhibitor KK-36-105.

FIG. 13C shows the inhibition constants (K_{I}, k_{inact}) of irreversible inhibitor KK-36-70.

FIG. 13D shows the inhibition constants (K_{I}, k_{inact}) of irreversible inhibitor KK-36-111.

FIG. 13E shows the inhibition constants (K_{I}, k_{inact}) of irreversible inhibitor KK-36-25.

FIG. 14A and FIG. 14B show selected changes in chemical shift for resonances in the ¹⁵N-¹H HSQC NMR spectrum of ERGi upon addition of KK-36-25.

FIG. 15A shows a surface representation of the structure of ERGi with the autoinhibition elements colored green, the DNA interaction surface colored cyan, and the residues where chemical shift changes were observed upon addition of KK-36-25 colored in red.

FIG. 15B shows a 180 degree rotation of FIG. 15A.

FIG. 16A shows the results of treatment of Ewing's sarcoma cell lines (in red) as well as two neuroblastoma cell lines (green) and a rhabdomyosarcoma cell line (blue) with the bivalent ERG inhibitor compound KK-36-25.

FIG. 16B shows the results of treatment of Ewing's sarcoma cell lines (in red) as well as two neuroblastoma cell lines (green) and a rhabdomyosarcoma cell line (blue) with the bivalent ERG inhibitor compound KK-36-70.

FIG. 16C shows the results of treatment of Ewing's sarcoma cell lines (in red) as well as two neuroblastoma cell lines (green) and a rhabdomyosarcoma cell line (blue) with the bivalent ERG inhibitor compound KK-36-84.

FIG. 16D shows the results of treatment of Ewing's sarcoma cell lines (in red) as well as two neuroblastoma cell lines (green) and a rhabdomyosarcoma cell line (blue) with the bivalent ERG inhibitor compound KK-36-111.

FIGS. 17A-17D show the effects of the ERG inhibitors on several leukemia cell lines.

FIGS. 18A-18C show the effects of the ERG inhibitors on several leukemia cell lines.

FIG. 19 shows the effects of the ERG inhibitors on one leukemia cell line.

FIG. 20 shows overexpression of FLI1 in acute myeloid leukemia (AML) cells.

FIG. 21 shows overexpression of ERG in acute myeloid leukemia (AML) cells.

FIG. 22 shows that ERG inhibitor KK-36-25 is selective for leukemia cell lines dependent on ERG.

FIG. 23A shows compound KK-36-25 effect on prostate cancer cell lines.

FIG. 23B shows compound KK-36-70 effect on prostate cancer cell lines.

FIG. 23C shows compound KK-36-84 effect on prostate cancer cell lines.

FIG. 23D shows compound KK-36-111 effect on prostate cancer cell lines.

FIG. 24 shows the effects of ERG inhibitors KK-36-25 and KK-36-84 on the expression of documented ERG target genes in an ERG fusion positive prostate cancer cell line (VCaP) and an ERG fusion negative prostate cancer cell line (DU145).

FIG. 25 shows the ERG overexpression observed in prostate cancer cells with the TMPRSS2-ERG fusion (^{˜}50% patients).

FIG. 26 shows the effects of KK-36-84 on two genes (via qPCR) that are activated by EWS-FLI1 in the Ewings sarcoma cell line A673.

FIG. 27 shows the effects of KK-36-84 on one gene (via qPCR) that is repressed by EWS-FLI1 in the Ewings sarcoma cell line A673.

FIG. 28A shows GSEA analysis of RNASeq data after treatment with KK-36-25 compared to dTAG degradation of EWS-FLI1 (12 hours, EWS502 cell line).

FIG. 28B shows GSEA analysis of RNASeq data after treatment with KK-36-84 compared to dTAG degradation of EWS-FLI1 (12 hours, EWS502 cell line).

FIG. 29 shows the heatmap of normalized enrichment scores (ssGSEA) for treatment effects of KK-36-84, KK-36-25, and dTAG degradation of EWS-FLI1 on a compendia of EWS/FLI gene sets.

FIG. 30 shows auto-inhibition and the stabilization of the auto-inhibited state by small molecules.

FIG. 31 shows polarized screening.

FIG. 32 shows bivalent inhibitors with a fragment targeting protein elements medicating auto-inhibition and a fragment that reacts covalently with ERG to improve potency and duration of action.

### DETAILED DESCRIPTION OF THE INVENTION

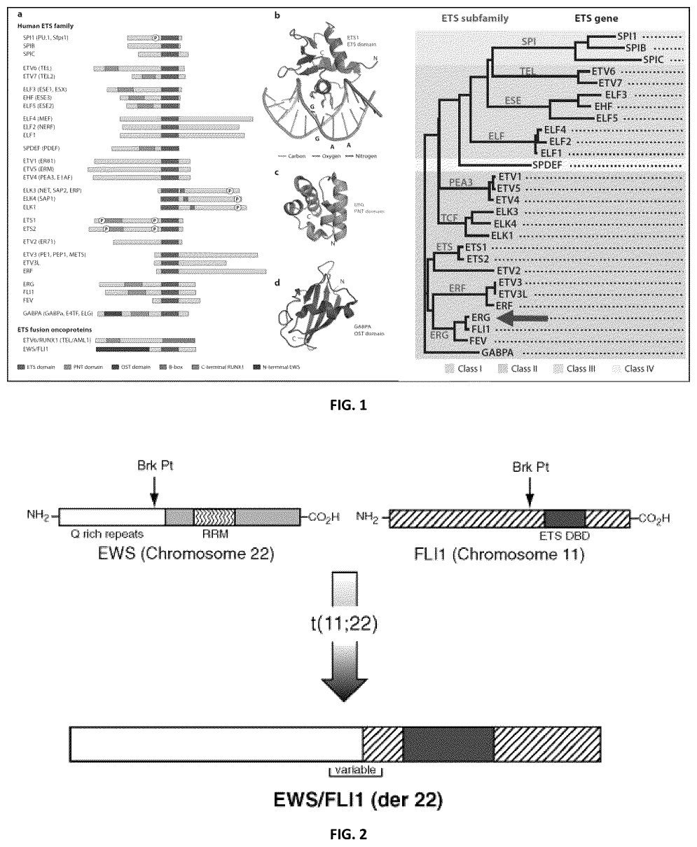

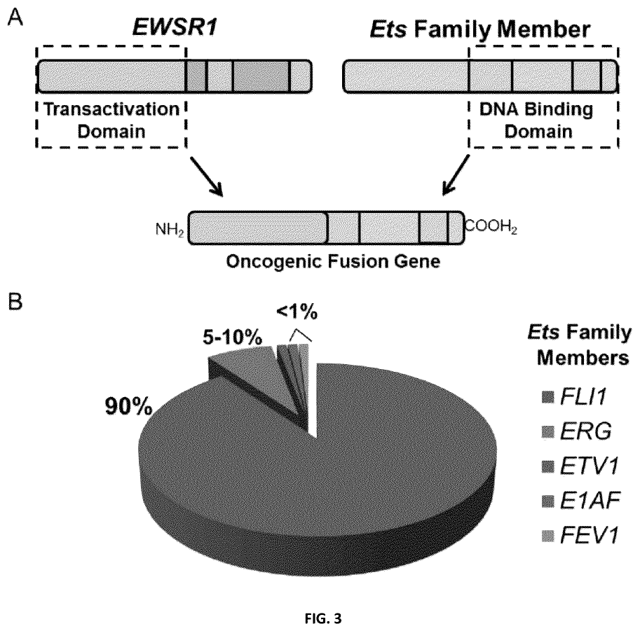

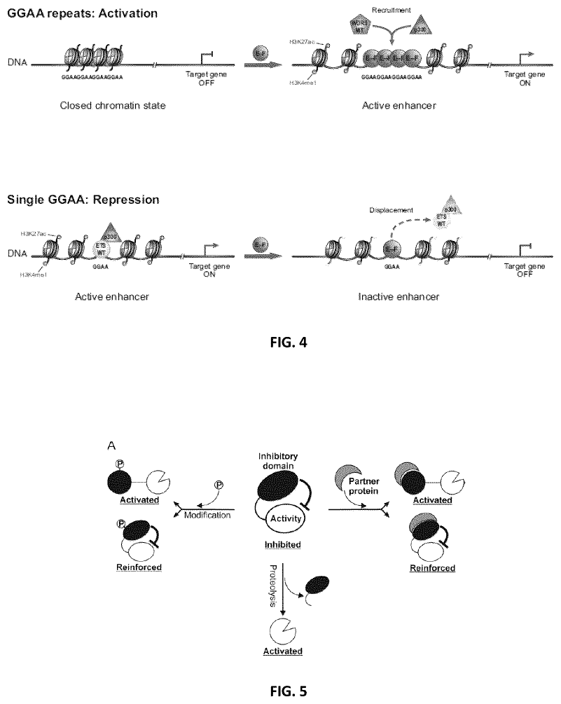

EWS-fusion oncoproteins in Ewing sarcoma. Ewing sarcoma is directly linked to a chromosomal translocation event between the EWS gene and a member of the ETS transcription factor family, most frequently FL11. FIG. 1 illustrates that ERG and FLI1 are members of the Ets transcription factor family (30). The resulting fusion protein, EWS-FLI1, is the dominant driver of Ewing sarcoma development and is required for disease maintenance and progression (1). FIG. 2 shows the EWS-FLI1 transcription factor schematically. Fusions with the ETS family member ERG are also observed in a subset of patients. FIG. 3 shows the oncogenic fusion gene of EWSR1 and the Ets family members. The (11;22)(q24;q12) translocation, which leads to expression of EWS-FL11, is identified in 85% of Ewing sarcoma cases. This fusion oncoprotein encodes a transcription factor translocation in which the DNA-binding domain (DBD) of the ETS transcription factor, FLI1 is fused to the transactivation domain of the EWSR1 gene, leading to aberrant gene expression. Analysis of EWS-FLI1 target gene promoters has revealed that EWS-FLI1 preferentially binds to repetitive GGAA-containing microsatellites in upregulated genes, and a study revealed that EWS-FLI1 reprograms gene regulatory circuits, acting as a pioneer factor. EWS-FLI1 multimers directly induce open chromatin and establish de novo enhancers at GGAA-containing microsatellite repeats that interact with promoters. EWS-FLI1 also inactivates conserved enhancers by displacing wildtype ETS from typical ETS sites. The Core Regulatory Circuitry (CRC) which interacts with, or independently of, EWS-FLI1 to govern gene expression in Ewing sarcoma cells, however, remains unknown. FIG. 4 shows the mechanisms of transcriptional regulation driven by EWS-FLI1 (6). The schematic illustrates the two distinct chromatin remodeling mechanisms underlying EWS-FL11-divergent transcriptional activity: enhancer induction and activation (top) with recruitment of WDR5 and p300 at GGAA repeats and enhancer repression (bottom) with displacement of endogenous ETS transcription factors and p300 at single GGAA canonical ETS motifs.

DNA binding is essential for the function of EWS-FL11. Early studies on EWS-FLI1 showed that binding to DNA was essential for the ability of the fusion protein to alter gene expression (2). The Ets domain of EWS-FLI1, which is the DNA binding domain, has been shown to be essential for the block in differentiation mediated by the fusion protein (3). Several studies have established the importance of binding of EWS-FLI1 to GGAA microsatellites for target gene regulation (4,5). A ChIP-Seq study revealed that EWS-FLI1 binds to GGAA-containing microsatellite repeats that interact with promoters and also displaces other ETS proteins from typical ETS sites (6), i.e., its ability to bind DNA is essential for its function. Based on all this data, the binding of EWS-FL1(ERG) to DNA is a valid target for therapeutic intervention.

ERG is also a driver in prostate cancer and leukemia. The Ets family member ERG has been linked to several cancers. ERG has been shown to be frequently over-expressed in prostate cancer (7). Perhaps more strikingly, ERG as well as other Ets family members have been shown to be the targets of chromosomal translocations with TMPRSS2 with the TMPRSS2-ERG fusion observed in approximately half of prostate cancer patient samples (8, 48). Indeed, the expression of TMPRSS2 is androgen regulated, resulting in over-expression of ERG or ETV1 in these prostate cancers. ERG is also the target of the t(16;21) in myeloid leukemia resulting in the TLS/FUS-ERG fusion protein (9) and is over-expressed in poor prognosis acute myeloid leukemias (10,11).

Dysregulation of gene expression is a hallmark of all cancers. It is critical for conferring stem cell like properties, such as self-renewal and chemo-resistance, on cancer cells. The specific gene expression program that confers these properties derives from aberrant activity of specific transcription factors which are drivers of disease. Clearly, the most direct and effective approach to alter this gene expression program is to directly target the activity of these transcription factors which are drivers of disease (transcription factor fusions EWS-FLI1 and EWS-ERG in the case of Ewing sarcoma). Transcription factors have traditionally been viewed as “undruggable” (except for nuclear hormone receptors) due to the need to target the more challenging protein-protein or protein-nucleic acid interactions through which these proteins act. There are still relatively few examples of such agents in the clinic, with the MDM2-p53 inhibitors being one example of such an agent that has progressed to the clinic (12-15). As there are few such agents, development of inhibitors targeting the EWS-FLI1 (ERG) fusion proteins is necessary.

Except for nuclear hormone receptors, pharma has long considered transcription factors to be “undruggable”. This is a direct result of long-held views that the protein-protein and protein-DNA interactions mediating transcription factor function are difficult to develop small molecule inhibitors for due to the properties of the binding surfaces. This is particularly true for inhibitors of protein-DNA binding for which there is a profound paucity of small molecule inhibitors. Drugging the DNA binding interface on such proteins, with their very high charge and convex surfaces, is a daunting task. The most effective way to inhibit the activity of a transcription factor is to reduce its binding to DNA thereby limiting the ability to bind target genes. However, this is problematic for DNA binding domains as they tend to have highly charged convex binding surfaces which are difficult to target with drug-like small molecules. A novel approach is proposed to achieve this, namely small molecule stabilization of auto-inhibition to inhibit transcription factor activity. Such an approach has not been applied to transcription factors, thus this represents a new paradigm for modulating transcription factor activity.

By targeting the auto-inhibitory modules of FLI1 and ERG, the concept that such an approach can achieve a high level of specificity in a family of related proteins (FLI1 and ERG are members of the 28 member Ets family) is supported. Demonstration of this concept is, particularly in the context of a family of transcription factors, novel.

Identification of Small Molecules that Bind Directly to the EWS-FLI1 or EWS-ERG Fusion Proteins to Modulate their DNA Binding

### Rationale

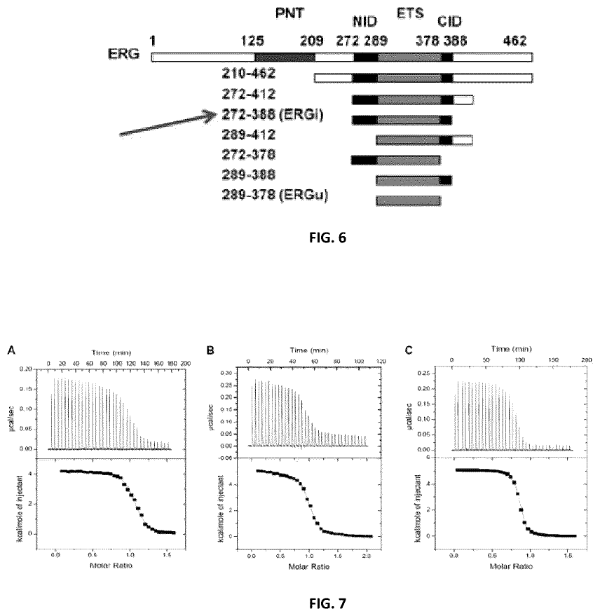

Auto-inhibition. As mentioned above, there is a profound paucity of small molecule inhibitors of protein-DNA interactions. A novel approach is proposed to target the DNA binding activity of the EWS and FL11 portions of EWS-FLI1 and EWS-ERG, namely the stabilization of their auto-inhibition. Auto-inhibition is a common property of many proteins, where regions outside a functional domain (catalytic domain, DNA binding domain, protein binding domain, etc.) bind to the functional domain to inhibit its activity (16). This process is often regulated by post-translational modifications or protein-protein interactions. Regions outside the DNA binding domain fold back onto the DNA binding domain to regulate activity (16). Auto-inhibition is modulated by partner protein binding as well as post-translational modification (phosphorylation) (16). Regions of protein mediating auto-inhibition are more “normal” in amino acid composition and potentially may present more favorable sites for drug-like small molecule interaction (16). FIG. 5 illustrates the principle of auto-inhibition. Auto-inhibition is a common property of many transcription factors, so this concept has the potential to have broad utility. In the context of families of transcription factors (like the ETS family to which FLI1 and ERG belong), which typically possess a highly conserved DNA binding domain present in all family members, this approach has a distinct advantage in terms of specificity. Namely, the sequences of the elements mediating auto-inhibition typically differ among family members, so targeting small molecules to these sites has the potential to achieve specificity for a specific transcription factor within a family of closely related proteins. Furthermore, these regions are comprised of a more typical distribution of amino acids, so the likelihood of finding drug-like molecules which can bind to these regions is higher.

ETS family of transcription factors. The ETS transcription factor family which includes FLI1 and ERG has 28 members defined by the presence of an ˜85 amino acid domain referred to as the Ets domain, which mediates sequence-specific DNA binding to a core DNA element GGAA/T. ETS family members have been implicated in many diseases including systemic lupus erythematosus, Down's syndrome, Ewing sarcoma, acute myeloid leukemia (AML), acute lymphocytic leukemia (ALL), rheumatoid arthritis, prostate cancer, and breast cancer, to name a few. For example, the ETS family member ERG has been linked to several cancers (prostate, Ewing sarcoma, and leukemia). ERG and the ETS protein ETV1 have been shown to be the targets of chromosomal translocations with TMPRSS2 observed in 80% of prostate cancer patient samples. Importantly for this application, fusions of EWS with the ETS family members FLI1 and ERG have been shown to be drivers of Ewing sarcoma.

Auto-inhibition of ETS family members. DNA binding by the ETS family members SAP-1, Elk-1, Net, Ets-1, Ets-2, and ERM has been shown to be auto-inhibited. The structural basis for auto-inhibition appears to differ among them as no homology is seen for the regions in the proteins that mediate auto-inhibition. Such auto-inhibition has been observed for other transcription factors such as p53, HSF, C/EBPβ, and RUNX1. Regulation of auto-inhibition occurs by means of interaction with other proteins as well as by specific phosphorylation.

ERG autoinhibition. The ETS family member ERG is regulated by auto-inhibition (17). FIG. 6 shows a schematic of the primary sequence of the ERG protein and of additional constructs of the ERG protein created to analyze autoinhibition (17). Table 1 shows the ERG construct ITC results for binding to DNA (17).

TABLE 1

| | | | | | | | | | | | Fold |
|---|---|---|---|---|---|---|---|---|---|---|---|
| Construct | Stoichiometry | | −TΔS (cal/mol) | | ΔH (cal/mol) | | ΔG (cal/mol) | | K_{D} (nmol) | | Inhibition |
| 1-462 | 1.05 | (±0.044) | −13369 | (±29) | 4205 | (±29.7) | −9164 | (±42) | 121 | (±5.01) | 3.3 |
| 210-462 | 1.1 | (±0.023) | −13572 | (±87) | 4303 | (±16.0) | −9269 | (±88) | 104 | (±5.86) | 2.8 |
| 272-412 | 0.94 | (±0.030) | −14181 | (±232) | 4900 | (±25.0) | −9281 | (±233) | 101 | (±8.22) | 2.7 |
| 272-388 | 0.98 | (±0.030) | −13891 | (±46) | 4924 | (±23.7) | −8967 | (±52) | 176 | (±10.4) | 4.8 |
| (ERGi) | | | | | | | | | | | |
| 289-412 | 0.98 | (±0.058) | −14732 | (±116) | 5062 | (±32.4) | −9670 | (±120) | 51 | (±10.4) | 1.4 |
| 272-378 | 0.99 | (±0.026) | −14645 | (±29) | 4731 | (±17.5) | −9914 | (±34) | 33 | (±2.50) | 0.9 |
| 289-388 | 0.99 | (±0.024) | −14819 | (±29) | 5024 | (±25.3) | −9795 | (±38) | 41 | (±3.67) | 1.1 |
| 289-378 | 1.04 | (±0.023) | −14703 | (±145) | 4851 | (±24.1) | −9852 | (±147) | 37 | (±2.77) | 1.0 |

(ERGu)

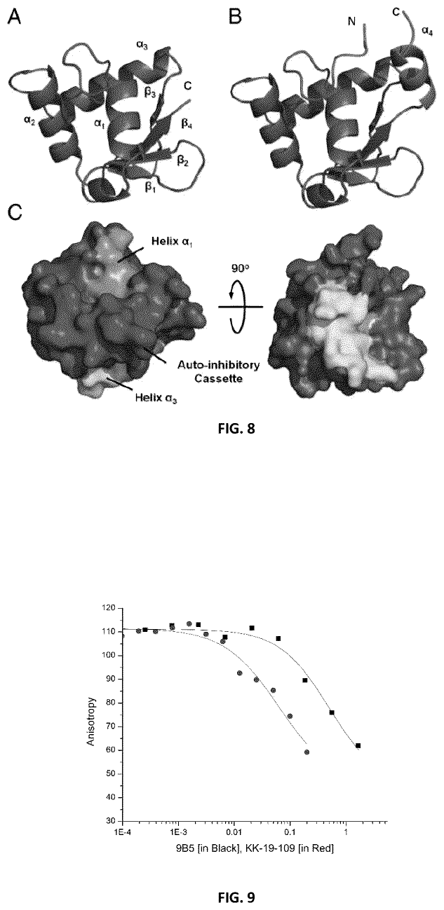

Stoichiometry, dissociation constant, and relative inhibition of ERG truncation constructs and point mutants. KD is given in nanomolar. Numbers in parenthesis are ±SE.
FIG. 7 shows isothermal titration calorimetry data for the binding of 3 of the constructs shown in FIG. 6 to DNA (A: ERG, B: ERGi, C: ERGu)(17). Structural studies showed that, like some other ETS family members, ERG auto-inhibition is mediated allosterically. Except for the change in rotamer of one Tyr residue, the structural changes in the Ets domain between the inhibited and uninhibited forms are subtle, suggesting that alteration of dynamics plays a key role in mediating auto-inhibition. FIG. 8 shows the 3D structures of ERGu (A) and ERGi (B) solved using x-ray crystallography and a surface representation of the structure of ERGi (C) (17). Recent NMR relaxation data from Kalodimos and co-workers has shown that dynamics in the DNA binding protein CAP is critical for optimal binding. The backbone dynamics of the auto-inhibited and uninhibited ERG Ets domain were characterized using NMR experiments and it was shown that the auto-inhibited Ets domain has dramatically reduced μs-ms timescale dynamics compared to the uninhibited ERG Ets domain. Changes in backbone dynamics between uninhibited and auto-inhibited Ets-1 have also been demonstrated using NMR, suggesting this is a key component of the mechanism of auto-inhibition. Because there are only 3 amino acid differences between ERG and FLI1 in the auto-inhibited construct of ERG that was identified, it is highly likely that FLI1 is auto-inhibited in the same manner. Importantly, the types 1 and 2 EWS-FLI1 fusions, which account for 83% of patient samples, include the protein regions mediating auto-inhibition (18,19). For EWS-ERG fusions, all types include the regions mediating auto-inhibition with the exception of the type 9e (20).

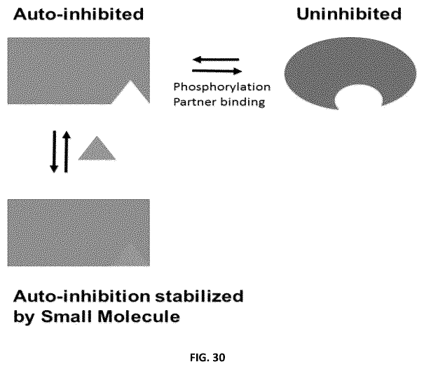

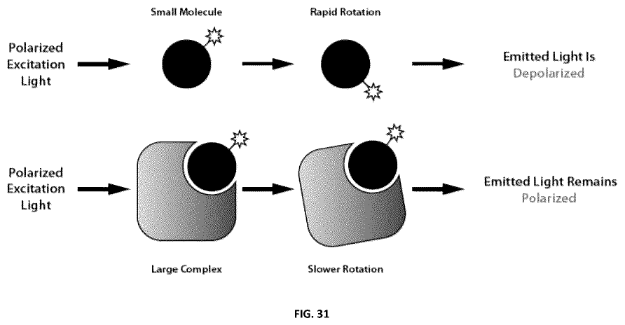

Small molecule modulation of auto-inhibition. Avery recent successful effort to develop a small molecule inhibitor of another class of “undruggable” target, the phosphatase SHP2, demonstrates the potential of targeting auto-inhibition to develop highly selective and potent inhibitors. Efforts to develop small molecule inhibitors targeting phosphatases have yielded relatively little progress, largely due to the nature of the active site and the inability to target that site with drug-like small molecules. The Novartis group instead screened for compounds that could stabilize the auto-inhibited state of the protein, optimize the activity of the initial hit, and show the structural basis for the stabilization of the auto-inhibited state (21). This serves as an effective proof-of-principle for the invention disclosed herein. Small molecules which stabilize auto-inhibition of the transcription factors FLI1 and ERG were developed to inhibit binding of EWS-FLI1 and EWS-ERG to DNA. FIG. 30 shows auto-inhibition and the stabilization of the auto-inhibited state by small molecules. Targeting auto-inhibition provides a path to get around targeting the protein-DNA interface and to achieve specificity. Unlike the highly conserved DNA binding domain (Ets domain) in the Ets family, the regions of the Ets family members mediating auto-inhibition are not conserved among family members, so compounds targeting these elements should be specific in their action. Because the regions of ETS family members that mediate auto-inhibition differ and show no sequence homology, molecules targeting this region in FLI1 and ERG are likely to be highly specific for these two virtually identical regions and not be active against other ETS family members.

### Preliminary Studies

### ERG Inhibitors

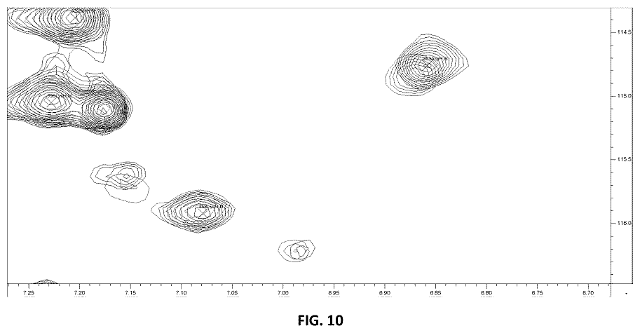

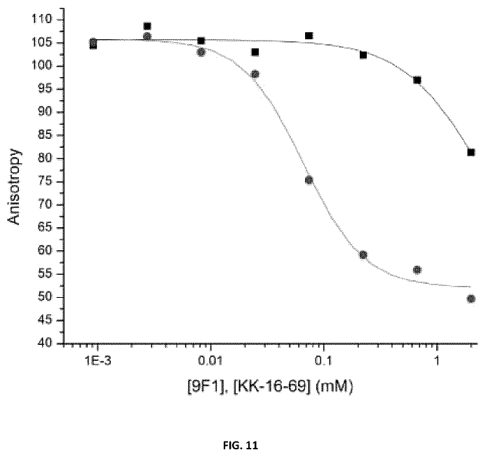

The constructs of ERG which retain full auto-inhibition were previously delineated (17). Fluorescence polarization-based assays for DNA binding were then developed, which were used for screening. In addition to the auto-inhibited form of ERG, it was important to also have an assay using its Ets domain, i.e., the uninhibited form of the protein, to compare action of compounds. Compounds which are active against the auto-inhibited form of the protein but have little or no effect on the uninhibited form were sought, indicating their binding is mediated by the non-conserved auto-inhibitory modules rather than the highly conserved Ets domain. A 12,000-compound fragment library was screened using the ERGi-DNA fluorescence polarization assay, as a high throughput screen with larger molecules failed to identify valid hits. FIG. 31 shows polarized screening. Fragment screening is an alternative approach that has gained a great deal of favor in the pharma industry recently that employs relatively small molecules (22). These molecules are significantly smaller than the compounds typically found in HTS collections, however they are highly drug-like making them good candidates for further elaboration. The auto-inhibited construct was screened first with a dose dependent screen of actives with ERGi using fluorescein- and Texas Red-DNA and then the positive hits were counter-screened with the ERG Ets domain, specifically a screen of actives with ERGu, the latter screen serving to remove compounds which bind to the conserved Ets domain or to DNA, which left 26 compounds which inhibit the auto-inhibited construct of ERG binding to DNA but not the uninhibited ERG (the Ets domain) binding to DNA. ERGi is the auto-inhibited construct of ERG (272-388) and ERGu is the uninhibited DNA binding domain construct of ERG (289-378). These 26 compounds were tested by NMR (¹⁵N-¹H HSQC spectra of the auto-inhibited ERG, specifically ERGi, with compounds) which resulted in the identification of 12 compounds which showed clear chemical shift changes in the ¹⁵N-¹H HSQC spectrum of ERG upon addition, indicating they are well-validated hits. The IC₅₀ of the validated hits was 0.6-7 mM. FIG. 9 shows a representative plot of FP assays for the fragments 9B5 (black/squares) and KK-19-109 (red/circles) with auto-inhibited ERG. FIG. 10 shows NMR chemical shift changes observed in an ¹⁵N-¹H HSQC NMR spectrum of ERGi alone and ERGi plus one of the active fragments. Importantly, chemical shift changes upon compound addition are observed in the ¹⁵N-¹H HSQC NMR spectra of ERG for amino acids located in the auto-inhibitory modules of ERG, indicating they do interact with these regions of the protein. It is critical to also develop assays for other ETS family members to assess the specificity of action of the compounds identified in the screen, so auto-inhibited constructs were expressed and assays were developed for five other ETS family members and assays for additional members of the family were also developed.

Using a standard medicinal chemistry approach, further optimization of the fragment hits were pursued. The specificity of parent fragments and their optimized derivatives with 5 additional Ets proteins which are representatives of 5 additional sub-families, were evaluated, as shown in Table 2. The data show excellent specificity for ERG and therefore clearly validate the hypothesis that targeting auto-inhibition will make it possible to achieve a high level of selectivity for ERG versus other ETS family members. Table 2 shows the results of IC₅₀ determinations for ERG auto-inhibited, ERG Ets domain (uninhibited), and auto-inhibited constructs of ELK1, ELF3, Ets-1, PU.1, and ETV6. NA in Table 2 represents no activity up to the 2000 μM maximum concentration. >number in Table 2 represents some activity at highest concentrations so data fit with a lower bound to obtain an estimate of IC₅₀.

TABLE 2

| | | | | | | | | |
|---|---|---|---|---|---|---|---|---|
| | | ERG | ERG | | | | | |
| | | Auto- | Isolated | | | | | |
| | | inhibited | Ets | | | | Spi1 | ETV6 |
| Cpd. | Protein | construct | Domain | ELK1 | ELF3 | Ets1 | (PU.1) | (TEL) |

| 9F1 | 2000 | NA | NA | NA | NA | NA | NA |
|---|---|---|---|---|---|---|---|
| | μM | | | | | | |
| KK-16-69 | 99 | NA | NA | NA | NA | >5700 | >7800 |
| | μM | | | | | μM | μM |
| 9B5 | 965 | NA | >2100 | 1830 | NA | >4100 | >2500 |
| | μM | | μM | μM | | μM | μM |
| KK-23-07 | 837 | NA | >2500 | 1740 | >2600 | >3300 | >4400 |
| | μM | | μM | μM | μM | μM | μM |
| 6H6 | 588 | NA | >4500 | NA | >4000 | NA | NA |
| | μM | | μM | | μM | | |
| KK-22-93 | 88 | >3000 | >4750 | >2000 | >3600 | NA | NA |
| | μM | μM | μM | μM | μM | | |

KK-23-07 has the following chemical structure:

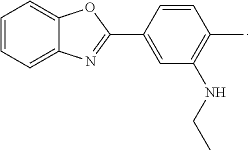 [SMILES: CCNc1cc(-c2nc3ccccc3o2)ccc1C]

This confirms that targeting of auto-inhibitory regions of protein is an effective approach to achieve specificity. These fragments are active with FLI1 as well.

### FLI1 Inhibitors

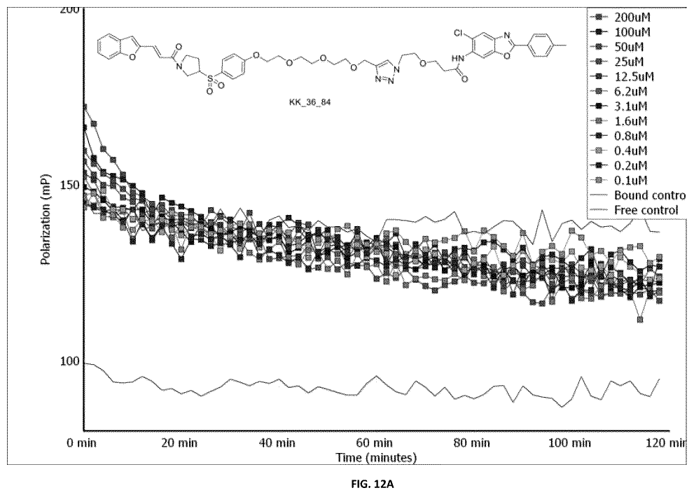

An auto-inhibited construct of FLI1 was expressed and purified based on the sequence identified for ERG. This FLI1 construct shows a very similar degree of auto-inhibition as observed for ERG, not surprisingly as there are only 3 amino acid differences between the two. Importantly, fragments identified from the ERG screen using FL11 were assayed and were shown to have similar activity, so they can also be used for development of FL11 inhibitors. FIG. 11 shows data from FP assay data for 9F1 and KK-16-69 with auto-inhibited FLI1 (same fragments shown in Table 2 above for auto-inhibited ERG).

Successful development of small molecule inhibitors of transcription factors and transcription factor fusions (CBFβ and CBFβ-SMMHC). The first targeted inhibitor for inv(16) leukemia which binds to the CBFβ-SMMHC transcription factor fusion protein and selectively disrupts its binding to RUNX1 to restore the RUNX1 driven gene expression program in inv(16) cells were developed (23). This inhibitor was shown to be selective for CBFβ3-SMMHC and that it did not impact CBFβ-RUNX binding, i.e., it shows selectivity for the leukemia inducing allele and has no effect on wildtype CBFβ. This inhibitor was shown to restore RUNX1 occupancy on target genes as well as gene expression for genes repressed by CBFβ-SMMHC. This inhibitor shows efficacy in a mouse model of inv(16) leukemia as well as against inv(16) patient cells. This represents one of a limited number of examples of successful targeting of a transcription factor for cancer treatment. Small molecule inhibitors of wildtype CBFβ-RUNX transcription factor binding have also been developed, which has been shown to alter RUNX occupancy on target genes and expression of RUNX regulated genes (24). These inhibitors show efficacy against leukemia (25), breast cancer (24), and ovarian cancer cells (49), consistent with known roles for RUNX in these cancers.

The invention relates to a compound of formula (I):

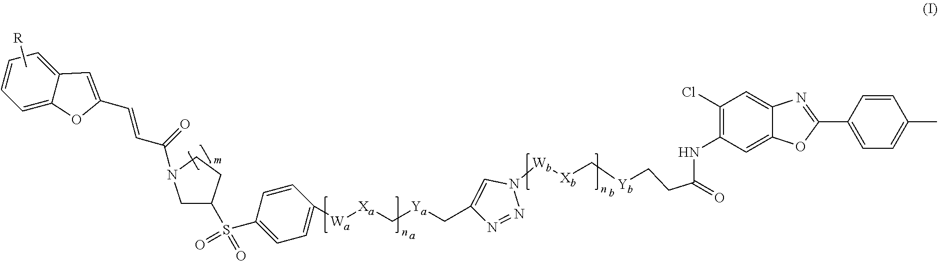

wherein:
R is, for each of the available binding sites, independently selected from the group consisting of H, F, Cl, Br, I, CH₃, OCH₃, CF₃, OCF₃, NO₂, NH₂, OH, N(CH₃)₂, CN, COCH₃, CONH₂, SO₂CH₃, and SO₂NH₂;
m is an integer from 1 to 4;
Wₐ, Xₐ, and Yₐ are for each occurrence independently O, CH₂, NH, cycloalkyl, benzyl, heterocycloalkyl, or heteroaryl, and one or more of Wₐ, Xₐ, and Yₐ are optionally not present, wherein the cycloalkyl, benzyl, heterocycloalkyl, and heteroaryl groups are independently unsubstituted or substituted, independently for each of the available binding sites, by H, F, Cl, Br, I, CH₃, OCH₃, CF₃, OCF₃, NO₂, NH₂, OH, N(CH₃)₂, CN, COCH₃, CONH₂, SO₂CH₃, or SO₂NH₂;
nₐ is an integer from 0 to 10;
W_{b}, X_{b}, and Y_{b} are for each occurrence independently O, CH₂, NH, cycloalkyl, benzyl, heterocycloalkyl, or heteroaryl, and one or more of W_{b}, X_{b}, and Y_{b} are optionally not present, wherein the cycloalkyl, benzyl, heterocycloalkyl, and heteroaryl groups are independently unsubstituted or substituted, independently for each of the available binding sites, by H, F, Cl, Br, I, CH₃, OCH₃, CF₃, OCF₃, NO₂, NH₂, OH, N(CH₃)₂, CN, COCH₃, CONH₂, SO₂CH₃, or SO₂NH₂; and
n_{b} is an integer from 0 to 10;
   or a pharmaceutically acceptable salt thereof.

In formula (I), R is, for each of the available binding sites, independently selected from the group consisting of H, F, Cl, Br, I, CH₃, OCH₃, CF₃, OCF₃, NO₂, NH₂, OH, N(CH₃)₂, CN, COCH₃, CONH₂, SO₂CH₃, and SO₂NH₂. R, for one or more available binding sites, may be H. R, for one or more available binding sites, may be F. R, for one or more available binding sites, may be Cl. R, for one or more available binding sites, may be Br. R, for one or more available binding sites, may be 1. R, for one or more available binding sites, may be CH₃. R, for one or more available binding sites, may be OCH₃. R, for one or more available binding sites, may be CF₃. R, for one or more available binding sites, may be OCF₃. R, for one or more available binding sites, may be NO₂. R, for one or more available binding sites, may be NH₂. R, for one or more available binding sites, may be OH. R, for one or more available binding sites, may be N(CH₃)₂. R, for one or more available binding sites, may be CN. R, for one or more available binding sites, may be COCH₃. R, for one or more available binding sites, may be CONH₂. R, for one or more available binding sites, may be SO₂CH₃. R, for one or more available binding sites, may be SO₂NH₂.

In formula (I), m is an integer from 1 to 4. For example, m is 1, 2, 3, or 4.

In formula (I), Wₐ, Xₐ, and Yₐ are for each occurrence independently O, CH₂, NH, cycloalkyl, benzyl, heterocycloalkyl, or heteroaryl, and one or more of Wₐ, Xₐ, and Yₐ are optionally not present, wherein the cycloalkyl, benzyl, heterocycloalkyl, and heteroaryl groups are independently unsubstituted or substituted, independently for each of the available binding sites, by H, F, Cl, Br, I, CH₃, OCH₃, CF₃, OCF₃, NO₂, NH₂, OH, N(CH₃)₂, CN, COCH₃, CONH₂, SO₂CH₃, or SO₂NH₂. Wₐ, Xₐ, and Yₐ for each occurrence may independently be 0. Wₐ, Xₐ, and Yₐ for each occurrence may independently be CH₂. Wₐ, Xₐ, and Yₐ for each occurrence may independently be NH. Wₐ, Xₐ, and Yₐ for each occurrence may independently be a cycloalkyl. Wₐ, Xₐ, and Yₐ for each occurrence may independently be a benzyl. Wₐ, Xₐ, and Yₐ for each occurrence may independently be a heterocycloalkyl. Wₐ, Xₐ, and Yₐ for each occurrence may independently be a heteroaryl. One or more of Wₐ, Xₐ, and Yₐ may optionally not be present. The cycloalkyl, benzyl, heterocycloalkyl, and heteroaryl groups are independently unsubstituted or substituted, independently for each of the available binding sites, by H, F, Cl, Br, I, CH₃, OCH₃, CF₃, OCF₃, NO₂, NH₂, OH, N(CH₃)₂, CN, COCH₃, CONH₂, SO₂CH₃, or SO₂NH₂.

In formula (I), nₐ is an integer from 0 to 10. For example, nₐ is 0, 1, 2, 3, 4, 5, 6, 7, 8, 9, or 10.

In formula (I), W_{b}, X_{b}, and Y_{b} are for each occurrence independently O, CH₂, NH, cycloalkyl, benzyl, heterocycloalkyl, or heteroaryl, and one or more of W_{b}, X_{b}, and Y_{b} are optionally not present, wherein the cycloalkyl, benzyl, heterocycloalkyl, and heteroaryl groups are independently unsubstituted or substituted, independently for each of the available binding sites, by H, F, Cl, Br, I, CH₃, OCH₃, CF₃, OCF₃, NO₂, NH₂, OH, N(CH₃)₂, CN, COCH₃, CONH₂, SO₂CH₃, or SO₂NH₂. W_{b}, X_{b}, and Y_{b} for each occurrence may independently be 0. W_{b}, X_{b}, and Y_{b} for each occurrence may independently be CH₂. W_{b}, X_{b}, and Y_{b} for each occurrence may independently be NH. W_{b}, X_{b}, and Y_{b} for each occurrence may independently be a cycloalkyl. W_{b}, X_{b}, and Y_{b} for each occurrence may independently be a benzyl. W_{b}, X_{b}, and Y_{b} for each occurrence may independently be a heterocycloalkyl. W_{b}, X_{b}, and Y_{b} for each occurrence may independently be a heteroaryl. One or more of W_{b}, X_{b}, and Y_{b} may optionally not be present. The cycloalkyl, benzyl, heterocycloalkyl, and heteroaryl groups are independently unsubstituted or substituted, independently for each of the available binding sites, by H, F, Cl, Br, I, CH₃, OCH₃, CF₃, OCF₃, NO₂, NH₂, OH, N(CH₃)₂, CN, COCH₃, CONH₂, SO₂CH₃, or SO₂NH₂.

In formula (I), n_{b} is an integer from 0 to 10. For example, n_{b} is 0, 1, 2, 3, 4, 5, 6, 7, 8, 9, or 10.

In formula (I), the compound of formula (I) may be a pharmaceutically acceptable salt thereof.

The term “cycloalkyl” as used herein refers to saturated or partially saturated, monocyclic, polycyclic, and spiro polycyclic carbocycle having 3-6 atoms per carbocycle. Illustrative examples of cycloalkyl groups as follows in the properly bonded moieties include:

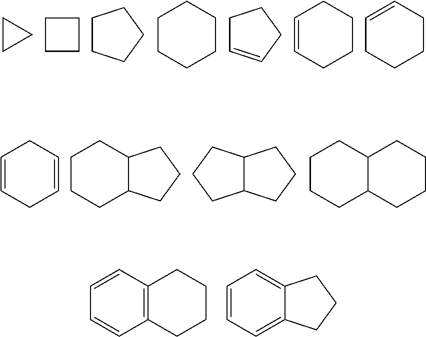

The term “heterocycloalkyl” as used herein refers to a monocyclic ring that is saturated or partially saturated and has 4-7 atoms selected from carbon atoms and up to two heteroatoms like nitrogen, sulfur, and oxygen monocyclic, polycyclic, and spiro polycyclic carbocycle having 3-6 atoms per carbocycle. Illustrative examples of heterocycloalkyl groups in the form of properly bonded moieties include:

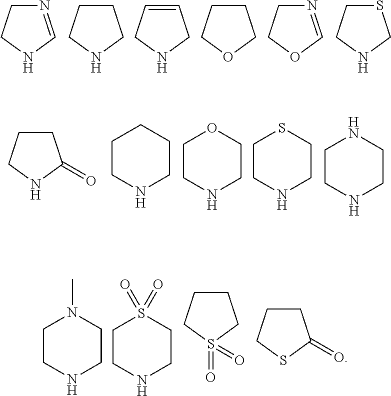

The term “heteroaryl” as used herein refers to monocyclic, fused bicyclic or polycyclic aromatic heterocycle consisting of ring atoms selected from carbon atoms and up to four heteroatoms like nitrogen, sulfur, and oxygen. Illustrative examples of heteroaryl groups in the form of properly bonded moieties include:

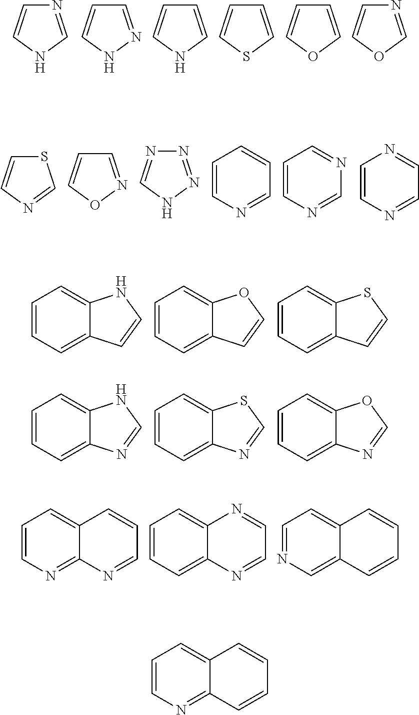

Exemplary compounds of formula (I) are those wherein R is hydrogen for each available binding site.

Other exemplary compounds are those wherein m is 1.

Other exemplary compounds are those wherein Wₐ and Yₐ are each 0 and Xₐ is CH₂.

Other exemplary compounds are those wherein one of Wₐ and X₂ is not present, the other of Wₐ and Xₐ is CH₂, and Yₐ is O.

Other exemplary compounds are those wherein nₐ is 1.

Other exemplary compounds are those wherein nₐ is 2.

Other exemplary compounds are those wherein nₐ is 3.

Other exemplary compounds are those wherein one of W_{b} and X_{b} is not present, the other of W_{b} and X_{b} is CH₂, and Y_{b} is O.

Other exemplary compounds are those wherein n_{b} is 1.

Exemplary compounds of the invention are found in Table 3.

TABLE 3

| Compound | Chemical Structure |
|---|---|
| KK-36-111 | 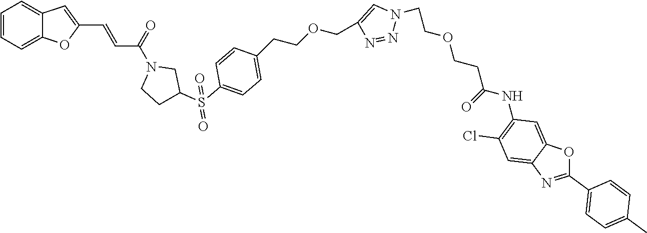 [SMILES: Cc1ccc(-c2nc3cc(Cl)c(NC(=O)CCOCCn4cc(COCCc5ccc(S(=O)(=O)C6CCN(C(=O)/C=C/c7cc8ccccc8o7)C6)cc5)nn4)cc3o2)cc1] |
| KK-36-70 | 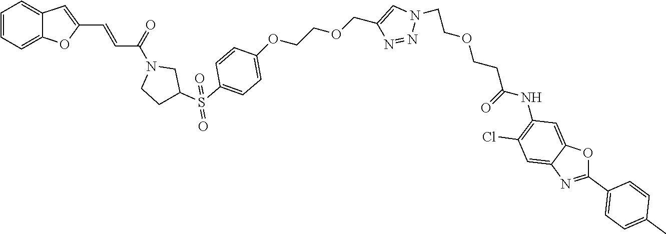 [SMILES: Cc1ccc(-c2nc3cc(Cl)c(NC(=O)CCOCCn4cc(COCCOc5ccc(S(=O)(=O)C6CCN(C(=O)/C=C/c7cc8ccccc8o7)C6)cc5)nn4)cc3o2)cc1] |
| KK-36-25 | 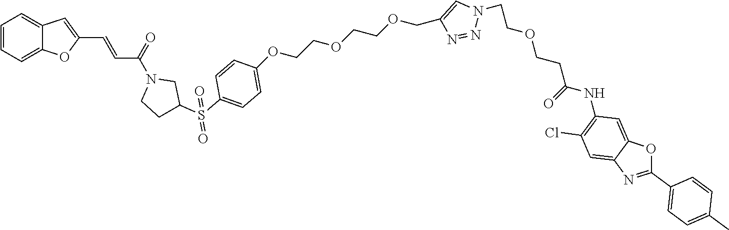 [SMILES: Cc1ccc(-c2nc3cc(Cl)c(NC(=O)CCOCCn4cc(COCCOCCOc5ccc(S(=O)(=O)C6CCN(C(=O)/C=C/c7cc8ccccc8o7)C6)cc5)nn4)cc3o2)cc1] |
| KK-36-84 | 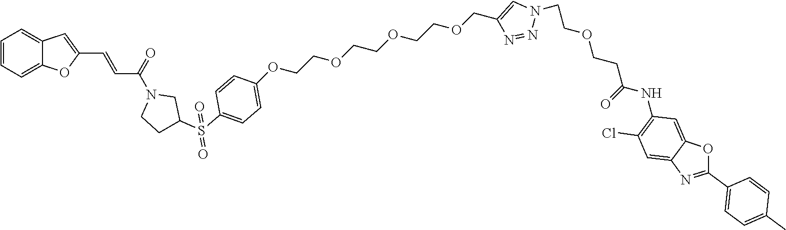 [SMILES: Cc1ccc(-c2nc3cc(Cl)c(NC(=O)CCOCCn4cc(COCCOCCOCCOc5ccc(S(=O)(=O)C6CCN(C(=O)/C=C/c7cc8ccccc8o7)C6)cc5)nn4)cc3o2)cc1] |

The invention further relates to a pharmaceutical composition comprising a compound of formula (I) and a pharmaceutically acceptable excipient. The compounds of formula (I) can be formulated as pharmaceutical compositions and administered to a mammalian host, such as a human patient in a variety of forms adapted to the chosen route of administration, i.e., orally or parenterally, by intravenous, intramuscular, topical, or subcutaneous routes.

The compounds of formula (I) may be systematically administered, e.g., orally, in combination with a pharmaceutically acceptable vehicle such as an inert diluent or an assimilable edible carrier, they may be enclosed in hard or soft shell gelatin capsules, may be compressed into tablets, or may be incorporated directly with the food of the patient's diet. For oral therapeutic administration, the compound of formula (I) may be combined with one or more excipients and used in the form of ingestible tablets, buccal tablets, troches, capsules, elixirs, suspensions, syrups, wafers, and the like. Such compositions and preparation should contain at least 0.1% of a compound of formula (I). The percentage of the compositions and preparations may be varied and may conveniently be between about 2 to about 60% of the weight of a given unit dosage form. The amount of compound of formula (I) in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the compound of formula (I). Sucrose or fructose as a sweetening agent, methyl and propylparabens as preservatives, a dye and flavoring such as cherry or orange flavor. Any material used in preparing any unit dosage form should be pharmaceutically acceptable and substantially non-toxic in the amounts employed. In addition, the compound of formula (I) may be incorporated into sustained-release preparations and devices.

The compound of formula (I) may also be administered intravenously or intraperitoneally by infusion or injection. Solutions of the compound of formula (I) or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile inject able or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the compound of formula (I) in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the compounds of formula (I) may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

The concentration of the compound(s) of formula (I) of the invention, in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%. The amount of the compound of formula (I), or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound of formula (I) is conveniently administered in unit dosage form; for example, containing 5 to 1000 mg, conveniently 10 to 750 mg, most conveniently, 50 to 500 mg of active ingredient per unit dosage form.

Ideally, the compound of formula (I) should be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four, or more sub-doses per day. The sub-dose itself may be further divided, e.g., into a number of discrete loosely spaced administrations; such as multiple injections or by direct or topical application.

The invention further relates to methods of treating cancer, comprising administering to a subject in need thereof a therapeutically effective amount of a compound of formula (I) or a pharmaceutical composition. For example, the invention provides a method of treating cancer, comprising administering to a subject in need thereof a therapeutically effective amount of a compound of formula (I). The invention also provides a method of treating cancer, comprising administering to a subject in need thereof a therapeutically effective amount of a pharmaceutical composition of the invention.

The cancer being treated may be Ewing sarcoma, leukemia, diffuse large B-cell lymphoma (DLBCL), and/or prostate cancer.

The compounds of formula (I), in particular those in Table 3, were tested for their effects on the growth of Ewing sarcoma cell lines as well as a rhabdomyosarcoma cell line as a control. The inhibitors are active against Ewing sarcoma cell lines with sub μM EC₅₀ values but not active with the rhabdomyosarcoma cell line. The relative activity of the compounds in the biochemical assay was also observed in the cellular data, consistent with an on-target mechanism of action. To further demonstrate on-target activity of the inhibitors, qPCR was used to assess the effects on two genes that are activated by EWS-FL11 (NROB1, NKX2-2) and one that is repressed by EWS-FLI1 (PHLDA1). The inhibitor decreases expression of the two genes activated by EWS-FL11 and activates the expression of the gene repressed by EWS-FLI1, consistent with an on-target mechanism of action. The inhibitors have also been evaluated across a panel of leukemia cell lines and see selective activity on cell lines with an ERG and/or FLI1 dependence. Additionally, testing of the inhibitors on prostate cancer cell lines shows selective activity for cell lines known to be driven by ERG and a reduction in expression of ERG target genes, which is again consistent with both selective activity of the inhibitors as well as an on-target mechanism of action. The most active molecule (KK-36-25) has K_{I}=6.3 μM and these compounds clearly demonstrate the time-dependent inhibition characteristic of irreversible inhibitors.

The invention also relates to process for preparing a compound of formula (I):

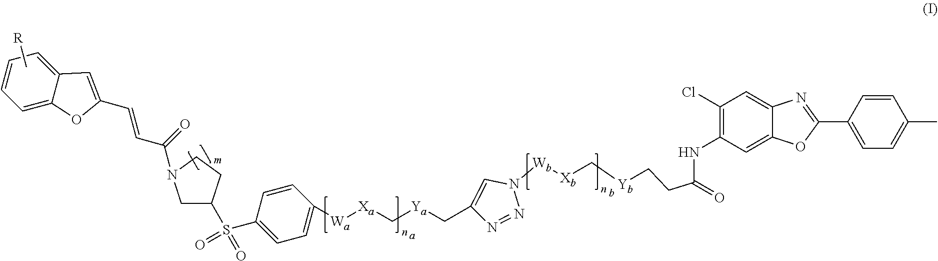

wherein in formula (I):
R is, for each of the available binding sites, independently selected from the group consisting of H, F, Cl, Br, I, CH₃, OCH₃, CF₃, OCF₃, NO₂, NH₂, OH, N(CH₃)₂, CN, COCH₃, CONH₂, SO₂CH₃, and SO₂NH₂;
m is an integer from 1 to 4;
Wₐ, Xₐ, and Yₐ are for each occurrence independently O, CH₂, NH, cycloalkyl, benzyl, heterocycloalkyl, or heteroaryl, and one or more of Wₐ, Xₐ, and Yₐ are optionally not present, wherein the cycloalkyl, benzyl, heterocycloalkyl, and heteroaryl groups are independently unsubstituted or substituted, independently for each of the available binding sites, by H, F, Cl, Br, I, CH₃, OCH₃, CF₃, OCF₃, NO₂, NH₂, OH, N(CH₃)₂, CN, COCH₃, CONH₂, SO₂CH₃, or SO₂NH₂;
nₐ is an integer from 0 to 10;
W_{b}, X_{b}, and Y_{b} are for each occurrence independently O, CH₂, NH, cycloalkyl, benzyl, heterocycloalkyl, or heteroaryl, and one or more of W_{b}, X_{b}, and Y_{b} are optionally not present, wherein the cycloalkyl, benzyl, heterocycloalkyl, and heteroaryl groups are independently unsubstituted or substituted, independently for each of the available binding sites, by H, F, Cl, Br, I, CH₃, OCH₃, CF₃, OCF₃, NO₂, NH₂, OH, N(CH₃)₂, CN, COCH₃, CONH₂, SO₂CH₃, or SO₂NH₂; and
n_{b} is an integer from 0 to 10;
   or a pharmaceutically acceptable salt thereof;
   comprising:
reacting a compound of formula (II) with a compound of formula (III) under conditions sufficient to form a compound of formula (IV):

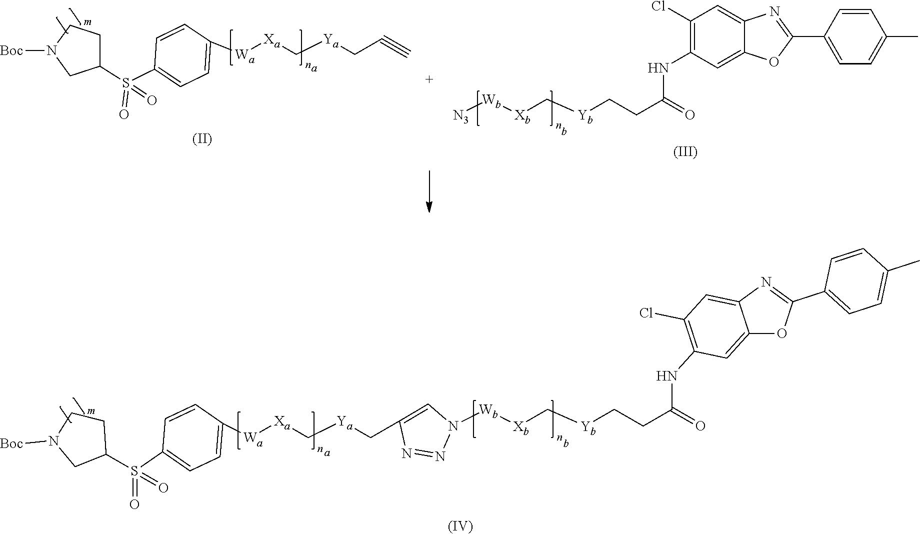

reacting the compound of formula (IV) under conditions sufficient to form a compound of formula (V):

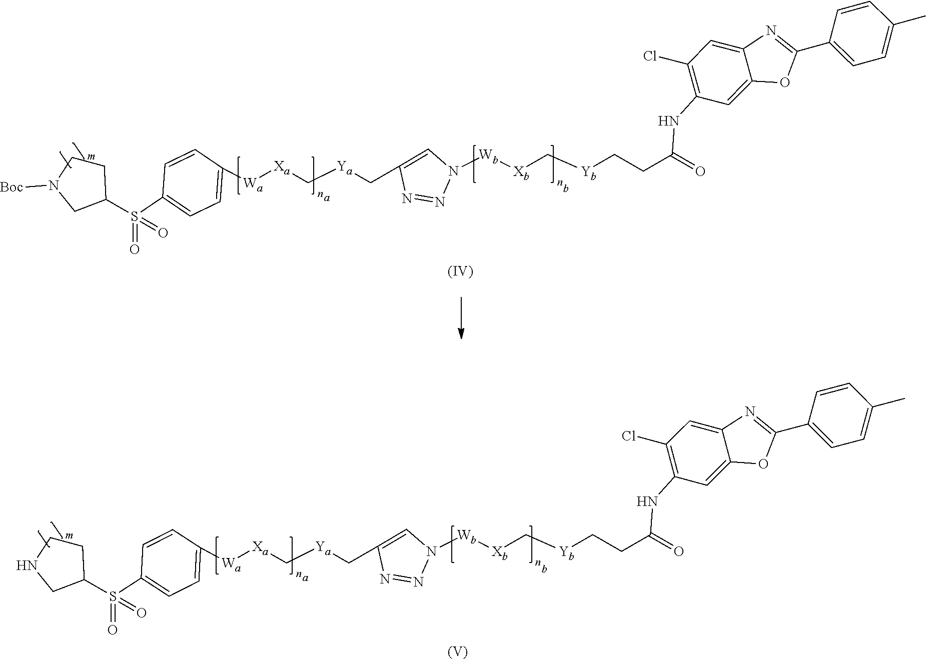

reacting the compound of formula (V) with a compound of formula (VI) under conditions sufficient to form the compound of formula (I):

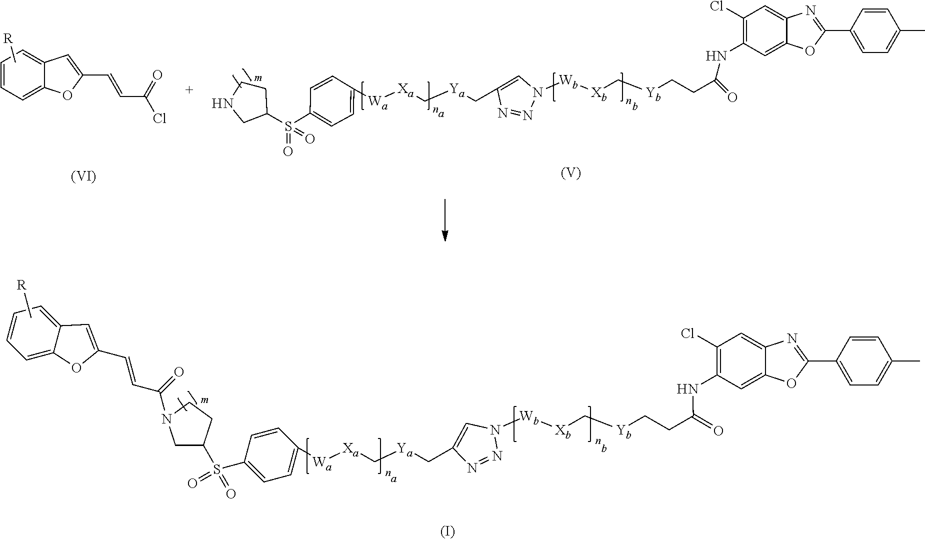

wherein R, m, Wₐ, Xₐ, Yₐ, nₐ, W_{b}, X_{b}, Y_{b}, and n_{b} in formulas (II), (III), (IV), (V), and (VI) are identical to how R, m, Wₐ, Xₐ, Yₐ, nₐ, W_{b}, X_{b}, Y_{b}, and n_{b} are defined in formula (I). By way of example, if Wₐ in formula (I) is chosen to be O, then Wₐ, for each of formulas (II), (IV), and (V), is 0 also. By way of another example, if Y_{b} in formula (I) is chosen to be CH₂, then Y_{b}, for each of formulas (III), (IV), and (V), is CH₂ also.

### EXEMPLARY EMBODIMENTS OF THE INVENTION

E1. A compound of formula (I):

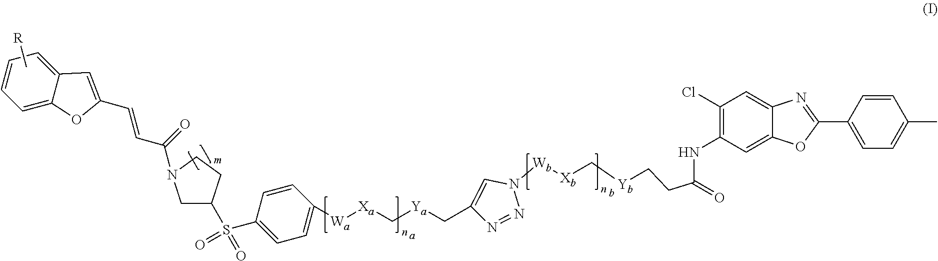

wherein:
R is, for each of the available binding sites, independently selected from the group consisting of H, F, Cl, Br, I, CH₃, OCH₃, CF₃, OCF₃, NO₂, NH₂, OH, N(CH₃)₂, CN, COCH₃, CONH₂, SO₂CH₃, and SO₂NH₂;
m is an integer from 1 to 4;
Wₐ, Xₐ, and Yₐ are for each occurrence independently O, CH₂, NH, cycloalkyl, benzyl, heterocycloalkyl, or heteroaryl, and one or more of Wₐ, Xₐ, and Yₐ are optionally not present, wherein the cycloalkyl, benzyl, heterocycloalkyl, and heteroaryl groups are independently unsubstituted or substituted, independently for each of the available binding sites, by H, F, Cl, Br, I, CH₃, OCH₃, CF₃, OCF₃, NO₂, NH₂, OH, N(CH₃)₂, CN, COCH₃, CONH₂, SO₂CH₃, or SO₂NH₂;
nₐ is an integer from 0 to 10;
W_{b}, X_{b}, and Y_{b} are for each occurrence independently O, CH₂, NH, cycloalkyl, benzyl, heterocycloalkyl, or heteroaryl, and one or more of W_{b}, X_{b}, and Y_{b} are optionally not present, wherein the cycloalkyl, benzyl, heterocycloalkyl, and heteroaryl groups are independently unsubstituted or substituted, independently for each of the available binding sites, by H, F, Cl, Br, I, CH₃, OCH₃, CF₃, OCF₃, NO₂, NH₂, OH, N(CH₃)₂, CN, COCH₃, CONH₂, SO₂CH₃, or SO₂NH₂; and
n_{b} is an integer from 0 to 10;
   or a pharmaceutically acceptable salt thereof.
E2. The compound of E1, wherein nₐ is an integer from 0 to 5.
E3. The compound of E1, wherein nₐ is an integer from 0 to 3.
E4. The compound of E1, wherein nₐ is 2.
E5. The compound of E1, wherein nₐ is 1.
E6. The compound of E1, wherein nₐ is 0.
E7. The compound of any of E1-E5, wherein Wₐ and Yₐ are each 0 and Xₐ is CH₂.
E8. The compound of any of E1-E5, wherein one of Wₐ and Xₐ is not present, the other of Wₐ and Xₐ is CH₂, and Yₐ is O.
E9. The compound of E1, wherein n_{b} is an integer from 0 to 3.
E10. The compound of E1, wherein n_{b} is 1.
E11. The compound of E9 or E10, wherein one of W_{b} and X_{b} is not present, the other of W_{b} and X_{b} is CH₂, and Y_{b} is 0.
E12. The compound of E1, wherein the compound is selected from the group consisting of:

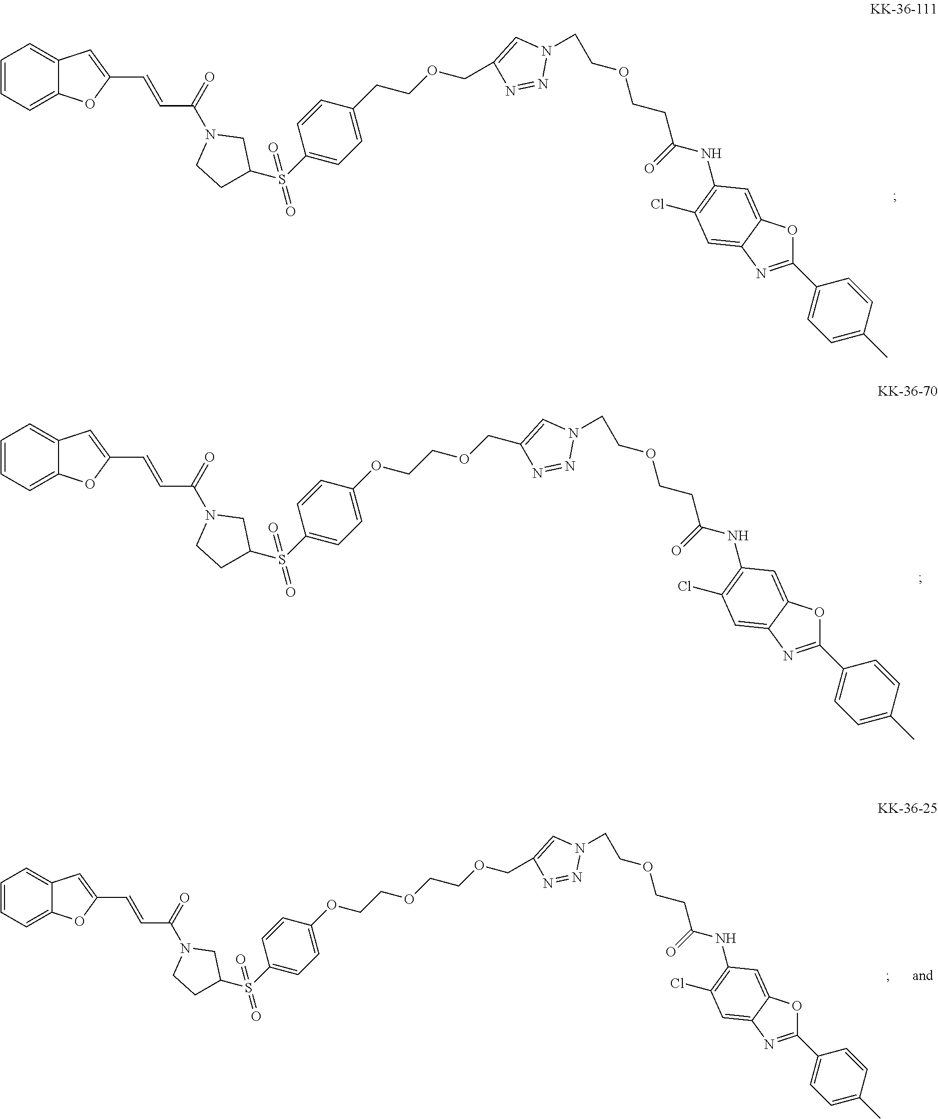 [SMILES: Cc1ccc(-c2nc3cc(Cl)c(NC(=O)CCOCCn4cc(COCCc5ccc(S(=O)(=O)C6CCN(C(=O)/C=C/c7cc8ccccc8o7)C6)cc5)nn4)cc3o2)cc1 ; Cc1ccc(-c2nc3cc(Cl)c(NC(=O)CCOCCn4cc(COCCOc5ccc(S(=O)(=O)C6CCN(C(=O)/C=C/c7cc8ccccc8o7)C6)cc5)nn4)cc3o2)cc1 ; Cc1ccc(-c2nc3cc(Cl)c(NC(=O)CCOCCn4cc(COCCOCCOc5ccc(S(=O)(=O)C6CCN(C(=O)/C=C/c7cc8ccccc8o7)C6)cc5)nn4)cc3o2)cc1]

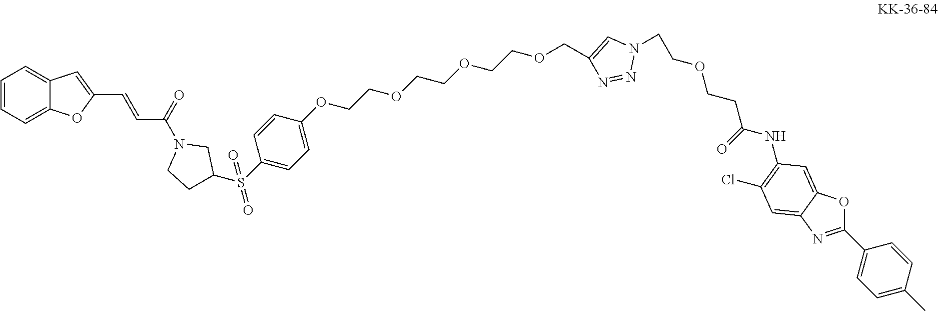 [SMILES: Cc1ccc(-c2nc3cc(Cl)c(NC(=O)CCOCCn4cc(COCCOCCOCCOc5ccc(S(=O)(=O)C6CCN(C(=O)/C=C/c7cc8ccccc8o7)C6)cc5)nn4)cc3o2)cc1]

E13. A pharmaceutical composition comprising a compound of any of E1-E12, and a pharmaceutically acceptable excipient.
E14. A method of treating cancer, comprising administering to a subject in need thereof a therapeutically effective amount of a compound of any one of E1-E12.
E15. A method of treating cancer, comprising administering to a subject in need thereof a therapeutically effective amount of a pharmaceutical composition of E13.
E16. The method of E14, wherein the cancer is selected from the group consisting of Ewing sarcoma, leukemia, diffuse large B-cell lymphoma (DLBCL), and prostate cancer.
E17. The method of E15, wherein the cancer is selected from the group consisting of Ewing sarcoma, leukemia, diffuse large B-cell lymphoma (DLBCL), and prostate cancer.
E18. A process for preparing a compound of formula (I):

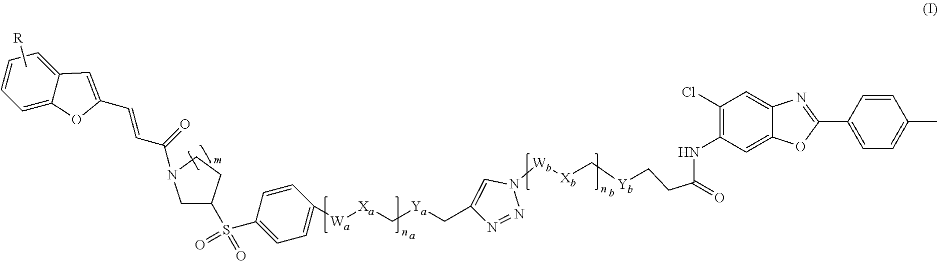

wherein in formula (I):
R is, for each of the available binding sites, independently selected from the group consisting of H, F, Cl, Br, I, CH₃, OCH₃, CF₃, OCF₃, NO₂, NH₂, OH, N(CH₃)₂, CN, COCH₃, CONH₂, SO₂CH₃, and SO₂NH₂;
m is an integer from 1 to 4;
Wₐ, Xₐ, and Yₐ are for each occurrence independently O, CH₂, NH, cycloalkyl, benzyl, heterocycloalkyl, or heteroaryl, and one or more of Wₐ, Xₐ, and Yₐ are optionally not present, wherein the cycloalkyl, benzyl, heterocycloalkyl, and heteroaryl groups are independently unsubstituted or substituted, independently for each of the available binding sites, by H, F, Cl, Br, I, CH₃, OCH₃, CF₃, OCF₃, NO₂, NH₂, OH, N(CH₃)₂, CN, COCH₃, CONH₂, SO₂CH₃, or SO₂NH₂;
nₐ is an integer from 0 to 10;
W_{b}, X_{b}, and Y_{b} are for each occurrence independently O, CH₂, NH, cycloalkyl, benzyl, heterocycloalkyl, or heteroaryl, and one or more of W_{b}, X_{b}, and Y_{b} are optionally not present, wherein the cycloalkyl, benzyl, heterocycloalkyl, and heteroaryl groups are independently unsubstituted or substituted, independently for each of the available binding sites, by H, F, Cl, Br, I, CH₃, OCH₃, CF₃, OCF₃, NO₂, NH₂, OH, N(CH₃)₂, CN, COCH₃, CONH₂, SO₂CH₃, or SO₂NH₂; and
n_{b} is an integer from 0 to 10;
   or a pharmaceutically acceptable salt thereof;
   comprising:
reacting a compound of formula (II) with a compound of formula (III) under conditions sufficient to form a compound of formula (IV):

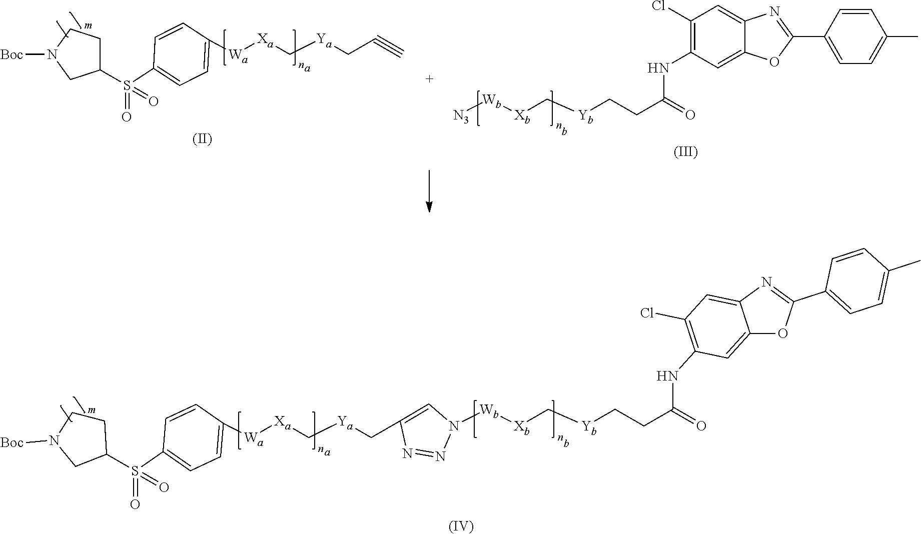

reacting the compound of formula (IV) under conditions sufficient to form a compound of formula (V):

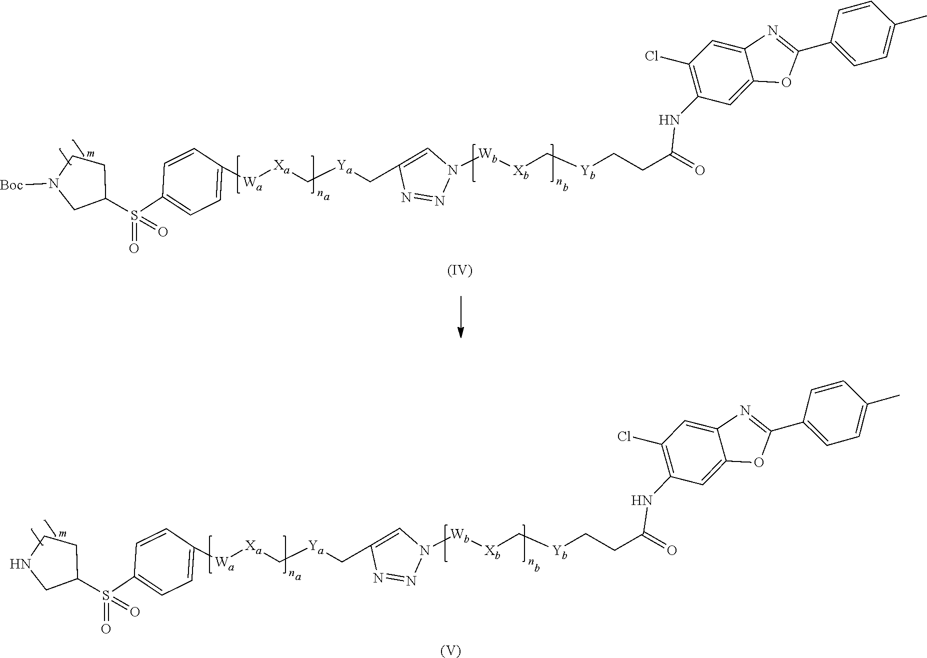

reacting the compound of formula (V) with a compound of formula (VI) under conditions sufficient to form the compound of formula (I):

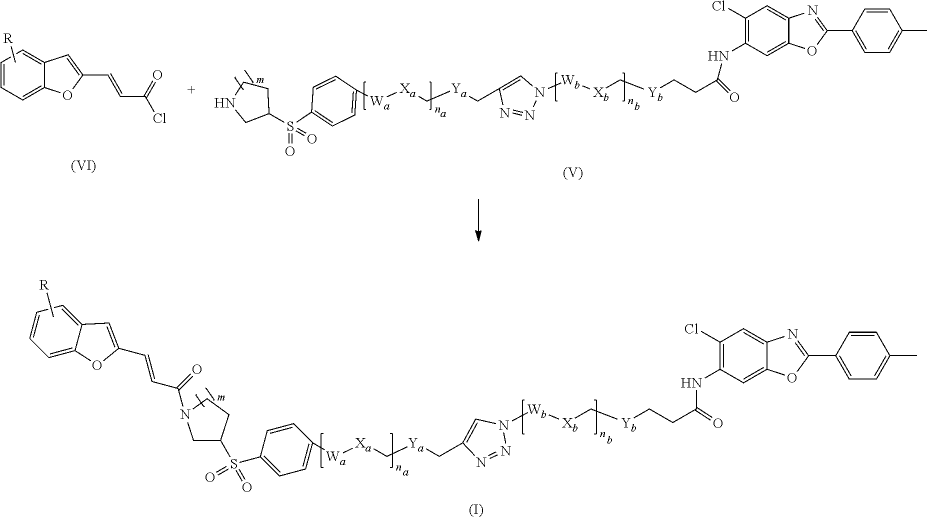

wherein R, m, Wₐ, Xₐ, Yₐ, nₐ, W_{b}, X_{b}, Y_{b}, and n_{b} in formulas (II), (Ill), (IV), (V), and (VI) are identical to how R, m, Wₐ, Xₐ, Yₐ, nₐ, W_{b}, X_{b}, Y_{b}, and n_{b} are defined in formula (I).
E19. The process of E18, wherein the compound of formula (I) is selected from the group consisting of:

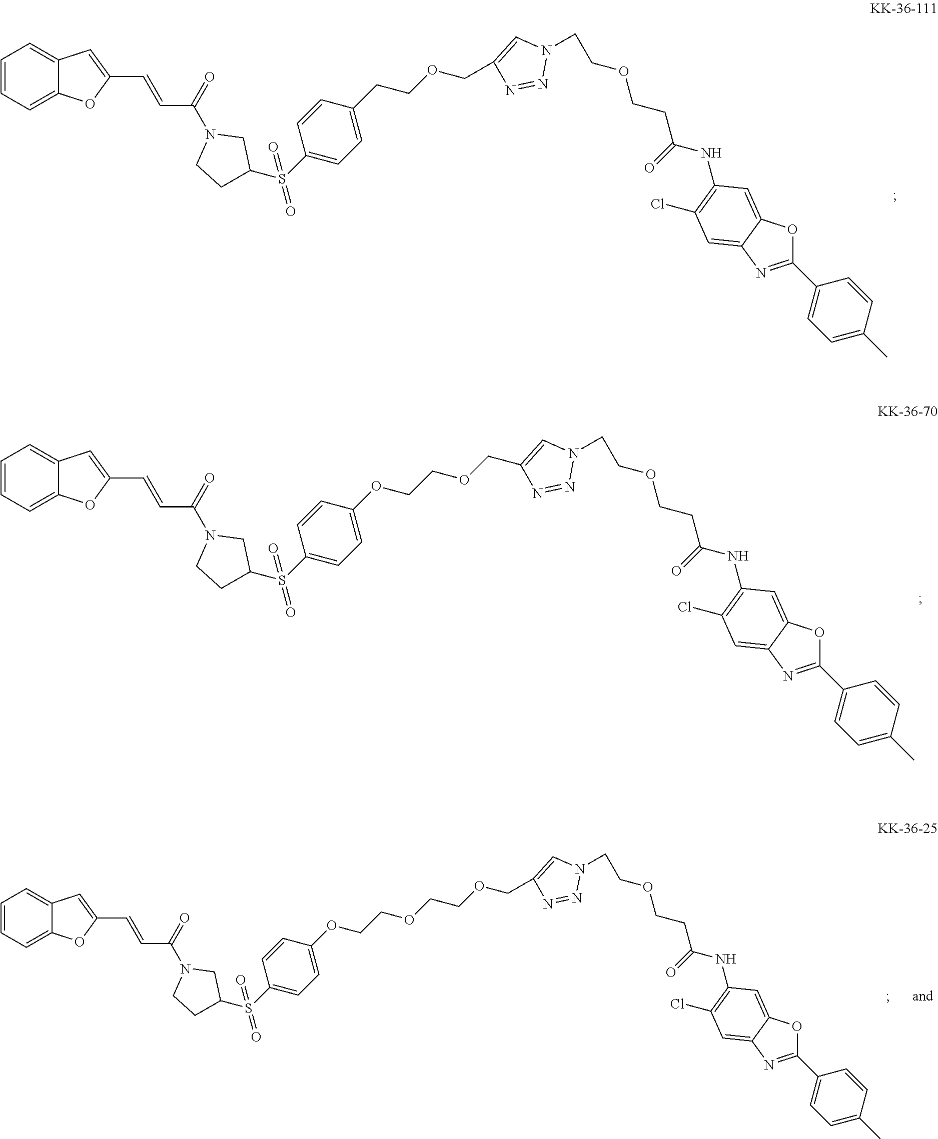 [SMILES: Cc1ccc(-c2nc3cc(Cl)c(NC(=O)CCOCCn4cc(COCCc5ccc(S(=O)(=O)C6CCN(C(=O)/C=C/c7cc8ccccc8o7)C6)cc5)nn4)cc3o2)cc1 ; Cc1ccc(-c2nc3cc(Cl)c(NC(=O)CCOCCn4cc(COCCOc5ccc(S(=O)(=O)C6CCN(C(=O)/C=C/c7cc8ccccc8o7)C6)cc5)nn4)cc3o2)cc1 ; Cc1ccc(-c2nc3cc(Cl)c(NC(=O)CCOCCn4cc(COCCOCCOc5ccc(S(=O)(=O)C6CCN(C(=O)/C=C/c7cc8ccccc8o7)C6)cc5)nn4)cc3o2)cc1]

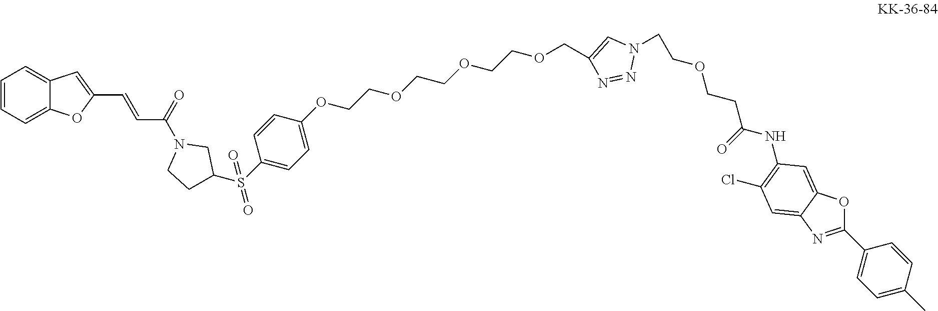 [SMILES: Cc1ccc(-c2nc3cc(Cl)c(NC(=O)CCOCCn4cc(COCCOCCOCCOc5ccc(S(=O)(=O)C6CCN(C(=O)/C=C/c7cc8ccccc8o7)C6)cc5)nn4)cc3o2)cc1]

### EXAMPLES

### Experimental Approach

### Optimization of Fragments

Optimization of fragment hits. Libraries of compounds were synthesized around the fragment hits to optimize activity and explore sites for linking to other fragments. These efforts led to fragments with IC₅₀ values of ca. 100 μM and identification of sites for linking on one of the classes of fragments.

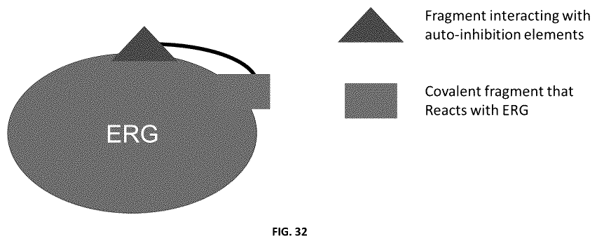

Screening of Cys reactive compound library. To improve the potency of the inhibitors it was hypothesized that linking the fragments identified to a molecule that could covalently react with ERG would drive the potency as well as potentially increase the duration of action. FIG. 32 shows bivalent inhibitors with a fragment targeting protein elements medicating auto-inhibition and a fragment that reacts covalently with ERG to improve potency and duration of action. A library of Cys reactive compounds were screened using the ERG-DNA FP assay to identify hits. Pre-reaction with ERG enhances activity of one autoinhibition fragment class. Two compound classes were identified for optimization and these were explored for sites to link to the current fragments.

Synthesis of and FP assays of bivalent inhibitors of ERG. Polyethylene glycol linkers and click chemistry were used to link the fragment to one of the reactive compounds with linkers of various lengths. The structures of the linked derivatives prepared are shown in Table 3 above, including KK-36-111, KK-36-70, KK-36-25, and KK-36-84. These compounds are ERG inhibitors, which are bivalent inhibitors with a fragment targeting autoinhibition and a fragment that reacts covalently with ERG. Scheme 1 shows the synthetic route for compounds KK-36-25, KK-36-70, and KK-36-84.

### Synthetic Scheme for K-36-25, K K-36-70, KK-36-84

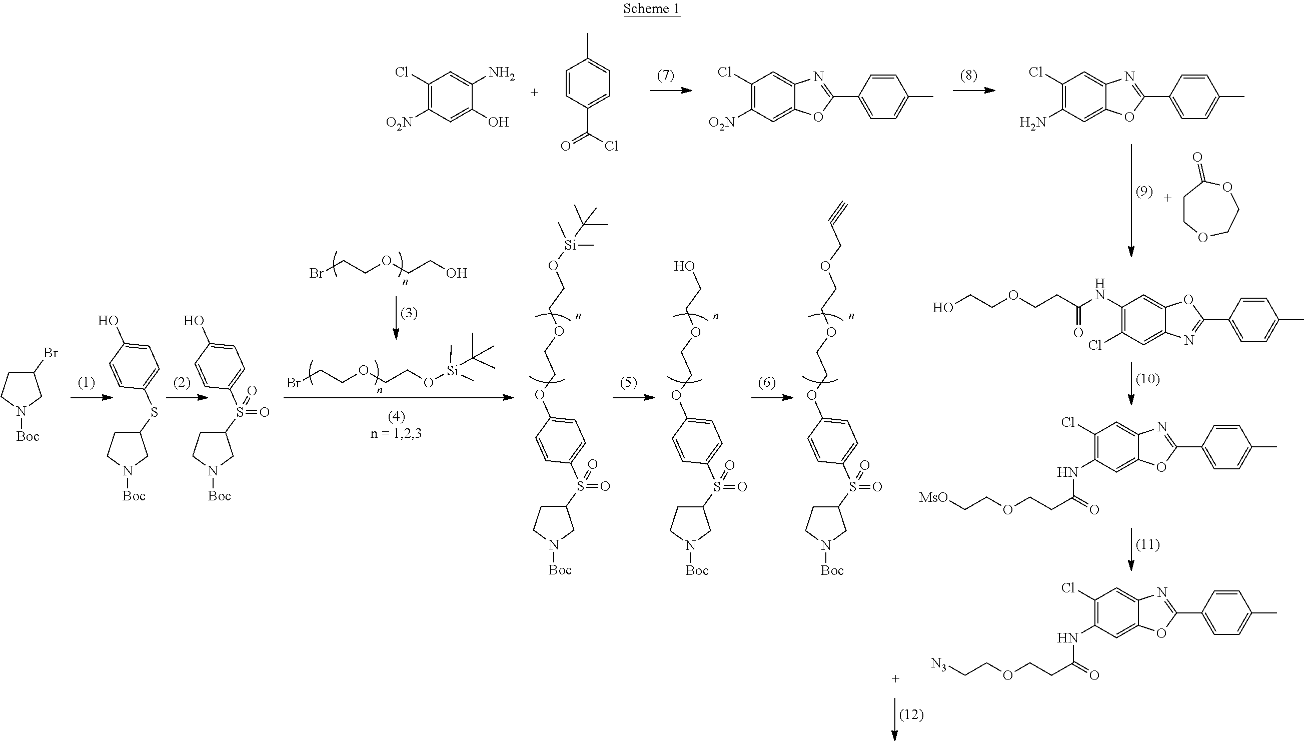

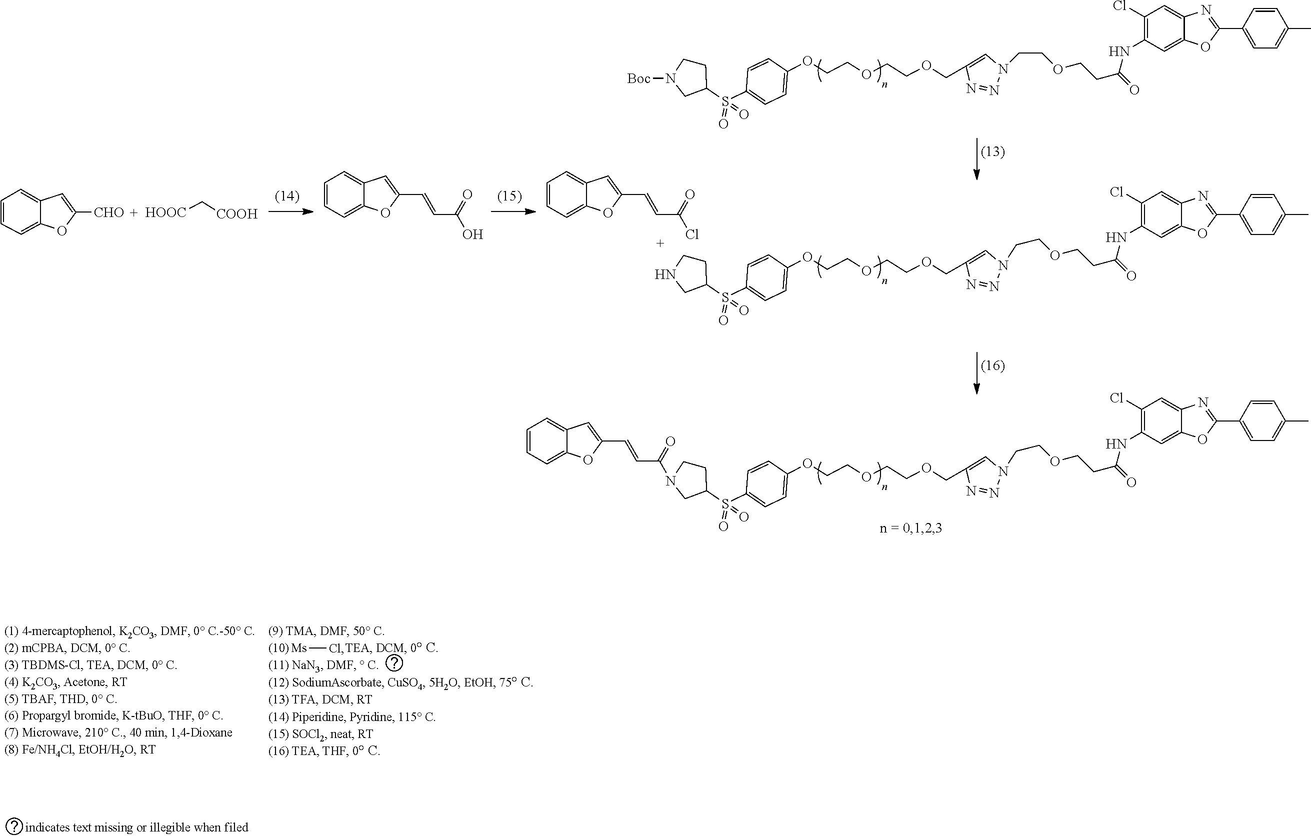

Scheme 2 shows the synthetic route for compound KK-36-111.

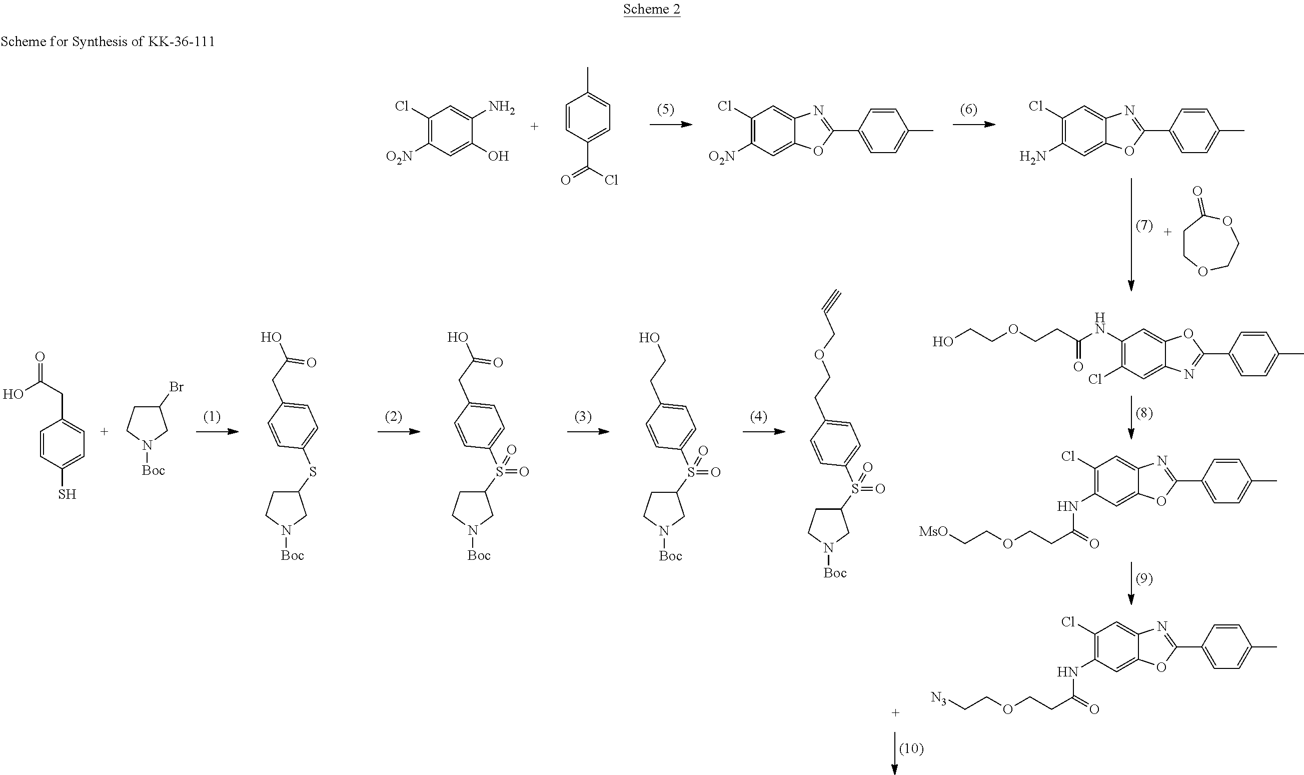

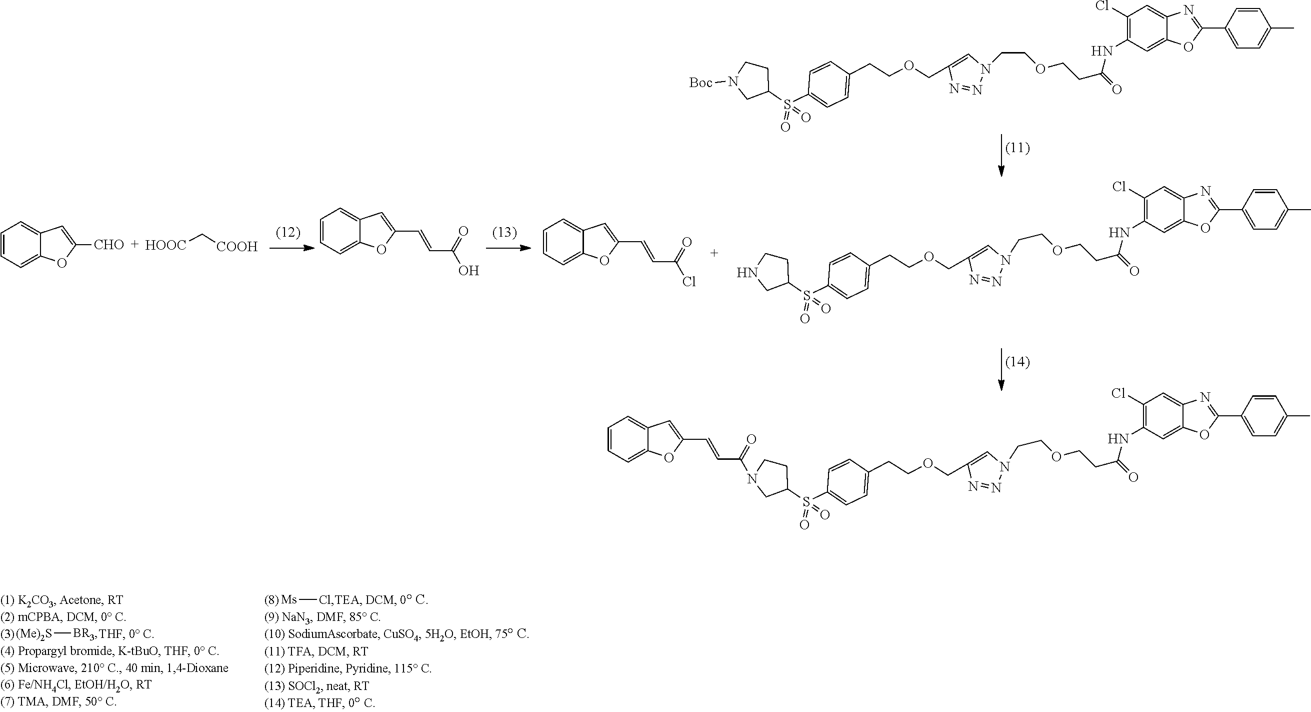

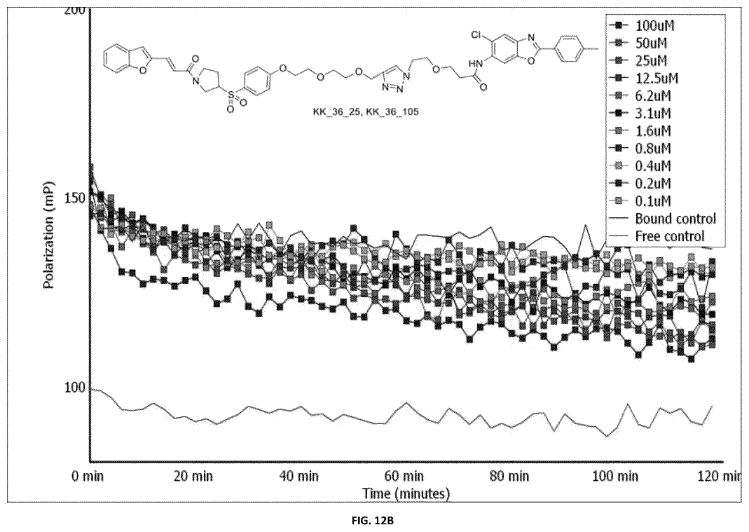

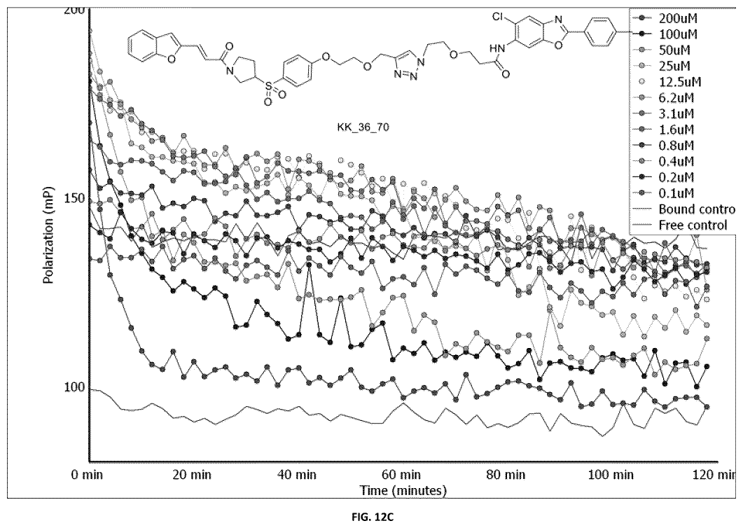

These compounds were assayed using the time-dependent ERG-DNA assay described above. Importantly, time-dependent inhibition was observed, a hallmark of irreversible inhibitors. FIG. 12A shows compound KK-36-84 assayed using time-dependent ERG-DNA assay. FIG. 12B shows compounds KK-36-25 and KK-36-105 assayed using the time-dependent ERG-DNA assay. FIG. 12C shows compound KK-36-70 assayed using the time-dependent ERG-DNA assay. FIG. 12D shows compound KK-36-111 assayed using the time-dependent ERG-DNA assay. ERG inhibitors show time-dependent inhibition characteristics of irreversible inhibitors based on the following formula:

$$Y = \frac{{K}_{inact} X}{{K}_{I} + X}$$

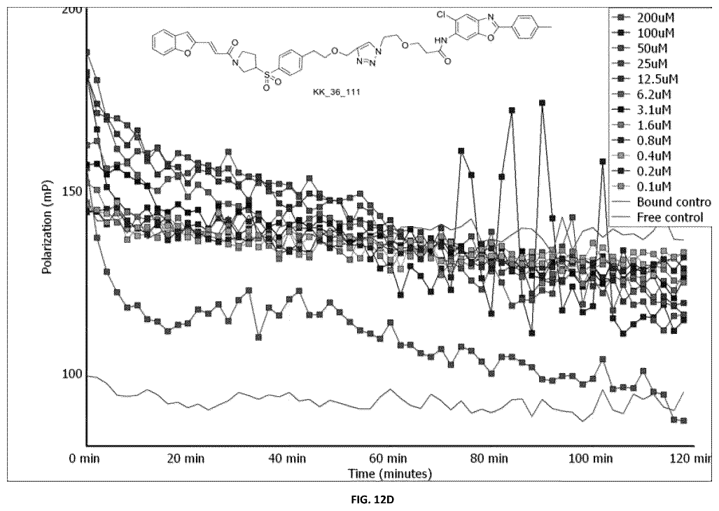

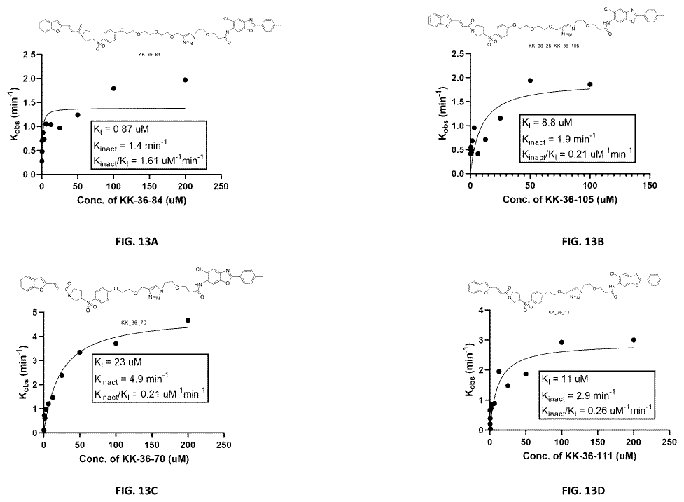

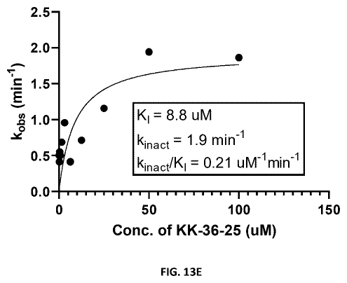

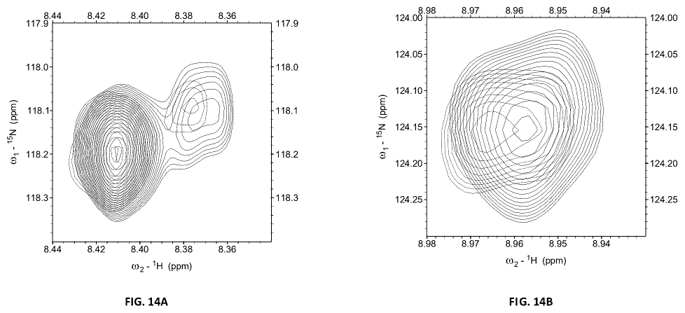

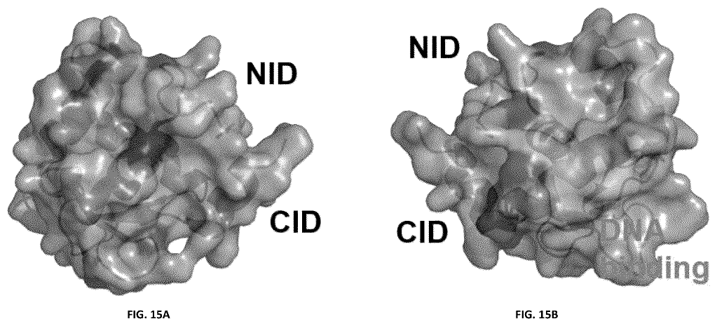

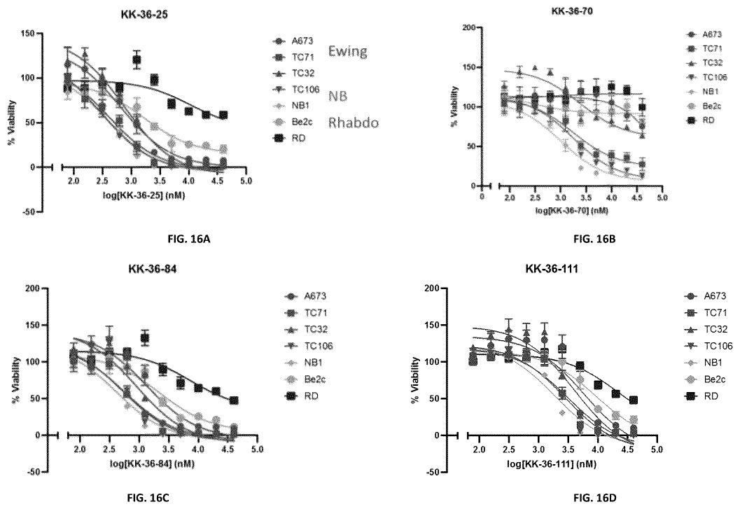

FIG. 13A shows the time dependent inhibition constants (K_{I}, k_{inact}) of irreversible inhibitor KK-36-84. FIG. 13B shows the time dependent inhibition constants (K_{I}, k_{inact}) of irreversible inhibitor KK-36-105. FIG. 13C shows the time dependent inhibition constants (K_{I}, k_{inact}) of irreversible inhibitor KK-36-70. FIG. 13D shows the time dependent inhibition constants (K_{I}, k_{inact}) of irreversible inhibitor KK-36-111. FIG. 13E shows the time dependent inhibition constants (K_{I}, k_{inact}) of irreversible inhibitor KK-36-25. K_{obs} values were determined from the initial slope for the first 15 minutes. NMR chemical shift perturbation data was consistent with 2 binding sites on ERG. FIG. 14A and FIG. 14B show selected changes in chemical shift for resonances in the ¹⁵N-¹H HSQC NMR spectrum of ERGi upon addition of KK-36-25. FIG. 15A shows surface representation of the structure of ERGi with the autoinhibition elements colored green, the DNA interaction surface colored cyan, and the residues where chemical shift changes were observed upon addition of KK-36-25 colored in red and FIG. 15B shows a 180 degree rotation of FIG. 15A.

Evaluation of ERG inhibitors on Ewing's sarcoma cell lines. FIGS. 16A-16D show the results of treatment of Ewing's sarcoma cell lines with the bivalent ERG inhibitors. FIG. 16A shows the results of treatment of Ewing's sarcoma cell lines with the bivalent ERG inhibitor compound KK-36-25. FIG. 16B shows the results of treatment of Ewing's sarcoma cell lines with the bivalent ERG inhibitor compound KK-36-70. FIG. 16C shows the results of treatment of Ewing's sarcoma cell lines with the bivalent ERG inhibitor compound KK-36-84. FIG. 16D shows the results of treatment of Ewing's sarcoma cell lines with the bivalent ERG inhibitor compound KK-36-111. Ewing's sarcoma has EWS-FLI1 and EWS-ERG fusions (FLI1 is highly homologous to ERG). ERG inhibitors show submicromolar EC50 values for Ewing's sarcoma cell lines. For the two most active compounds (KK-36-25, KK-36-84), IC₅₀ values for inhibition of the growth of the Ewing's sarcoma cell lines of 1 μM or lower were observed. Importantly, for the unrelated rhabdomyosarcoma cell line included in this panel, there was minimal inhibition of growth.

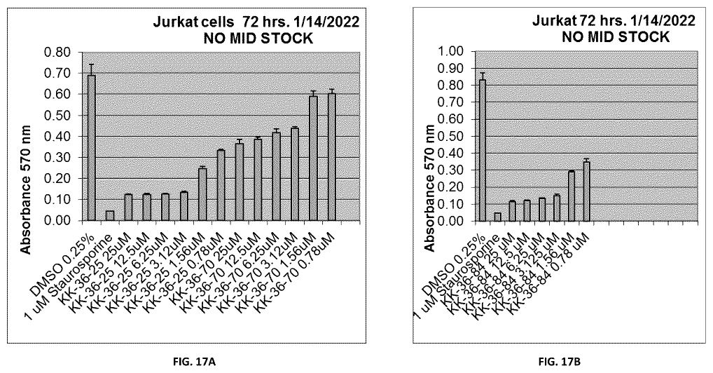

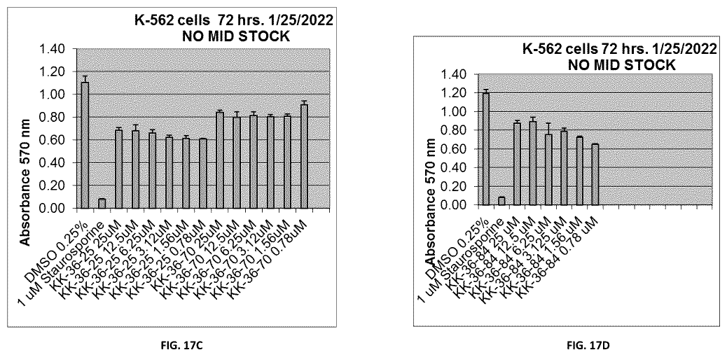

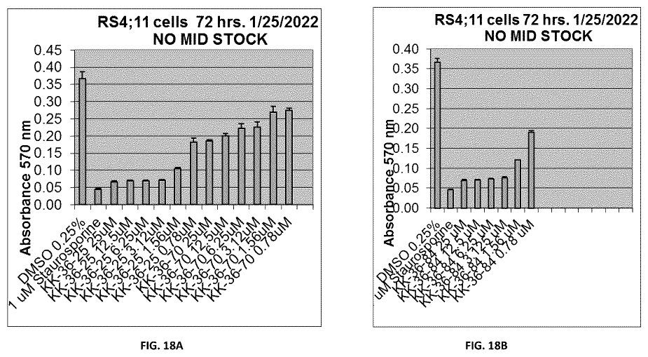

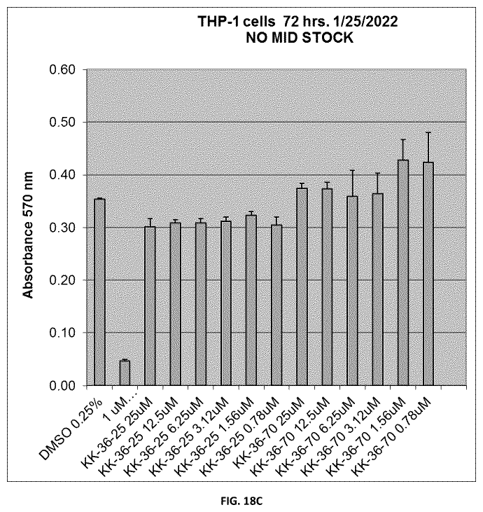

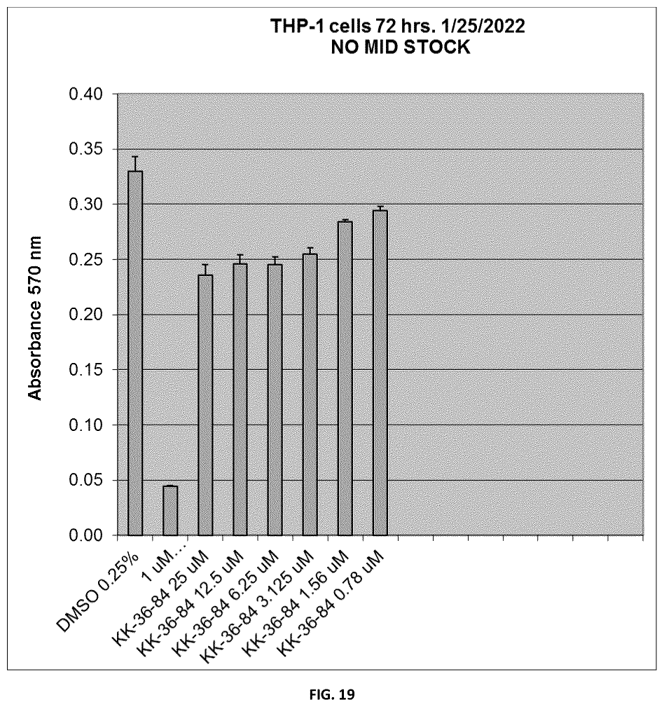

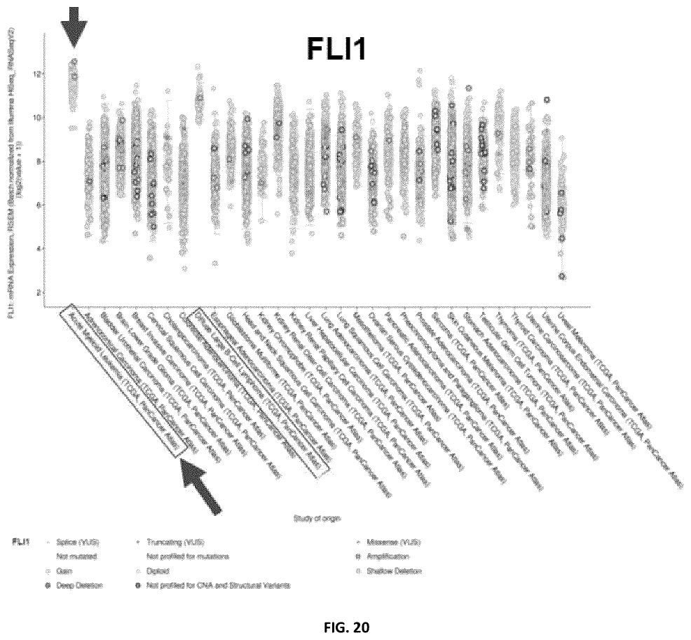

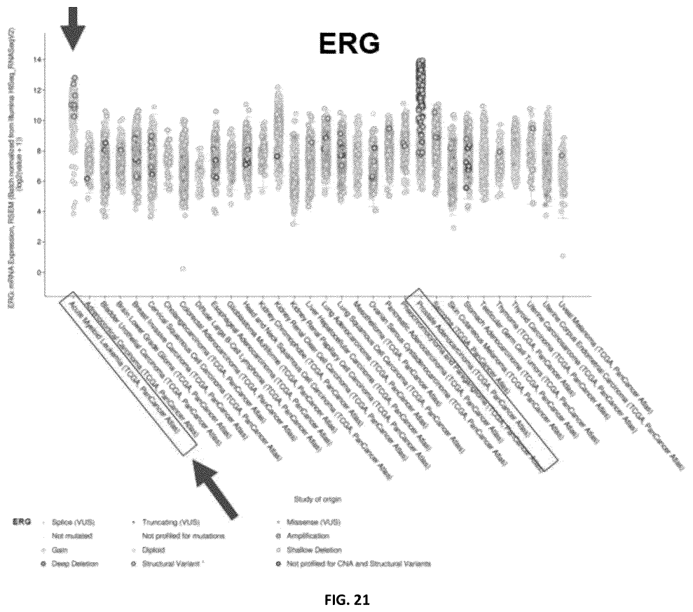

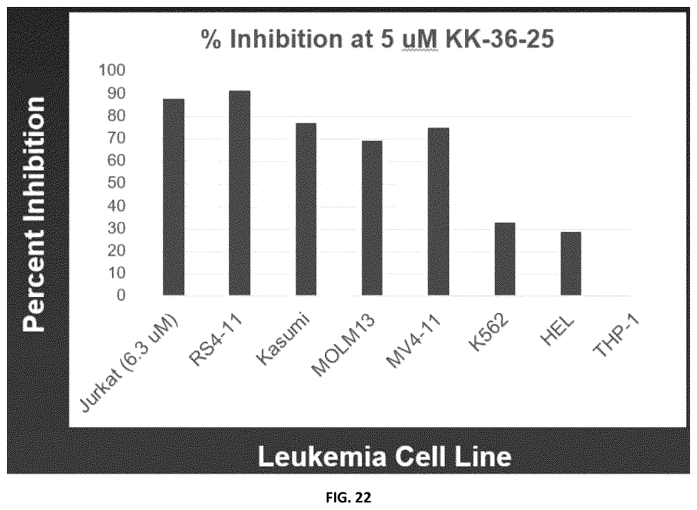

ERG inhibitor effects on leukemia cell lines. FIG. 17A-17D and FIG. 18A-18C show the effects of the ERG inhibitor on several leukemia cell lines. FIG. 19 shows the effects of the ERG inhibitors on one leukemia cell line. ERG inhibitors show efficacy against T-ALL cell line (Jurkat) and selective activity against specific leukemia cell lines. FIG. 20 shows overexpression of FLI1 in acute myeloid leukemia (AML) cells. FIG. 21 shows overexpression of ERG in acute myeloid leukemia (AML) cells. As expected from previous literature indicating a strong dependence on ERG for T-ALL, the T-ALL cell line Jurkat is quite sensitive to the ERG inhibitors (sub μM IC₅₀ values). K562 and THP-1 cell lines were relatively insensitive to the inhibitors whereas the RS4;11 cell line was sensitive, indicating differential dependence on ERG in different cell lines. FIG. 22 shows that ERG inhibitor KK-36-25 is selective for Leukemia cell lines dependent on ERG. Jurkat is ERG dependent; RS4-11 has high ERG; Kasumi is ERG dependent; K562 is low ERG; and THP-1 is low ERG.

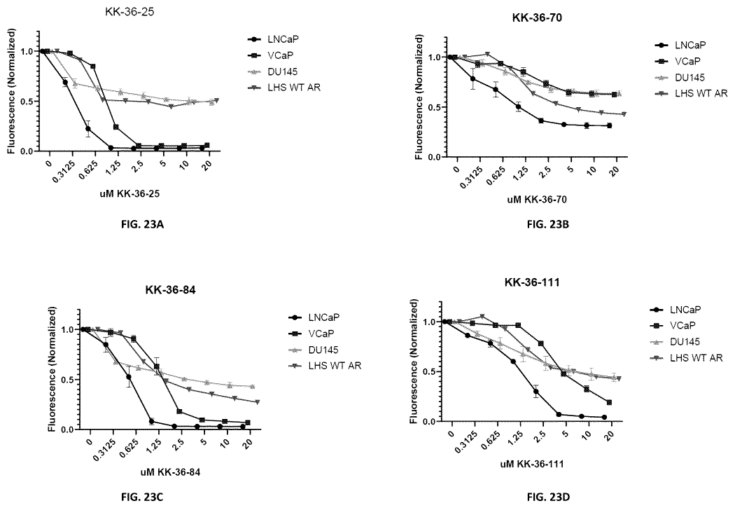

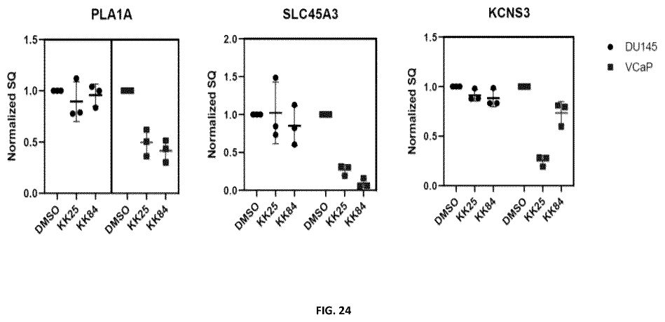

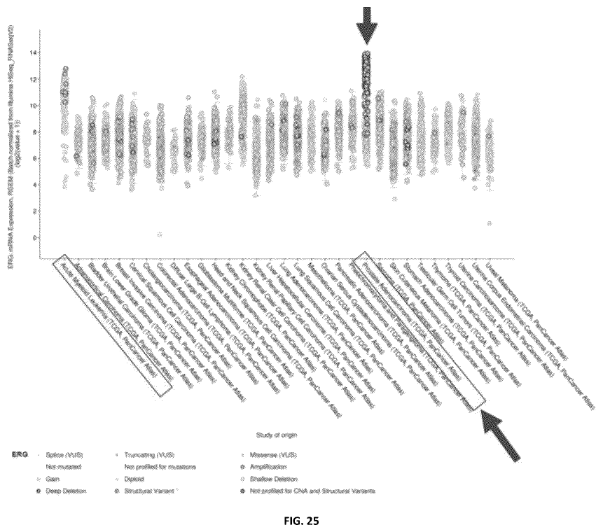

ERG inhibitor effects on prostate cancer cell lines. FIG. 23A shows compound KK-36-25 effect on prostate cancer cell lines. FIG. 23B shows compound KK-36-70 effect on prostate cancer cell lines. FIG. 23C shows compound KK-36-84 effect on prostate cancer cell lines. FIG. 23D shows compound KK-36-111 effect on prostate cancer cell lines. In FIGS. 23A-23D LHS represents n=1 and all others represent n=3. VCaP is ERG fusion; DU145 is brain met, no AR, not ERG dependent; and cellular efficacy correlates with results of biochemical assays. VCaP is prostate cell line with an ERG fusion and LNCaP is a prostate cancer cell line with an ETV1 fusion (ETV1 is a related Ets family member). Neither DU145 nor LHS WT AR harbor an Ets family member fusion. Consistent with this, the inhibitors show good activity against VCaP and LNCaP but quite limited activity against DU145 and LHS WT AR in terms of inhibiting growth. FIG. 24 shows the effects of ERG inhibitors KK-36-25 and KK-36-84 on the expression of documented ERG target genes in an ERG fusion positive prostate cancer cell line (VCaP) and an ERG fusion negative prostate cancer cell line (DU145). FIG. 25 shows the ERG overexpression observed in prostate cancer cells with TMPRSS2-ERG fusion (^{˜}50% patients).

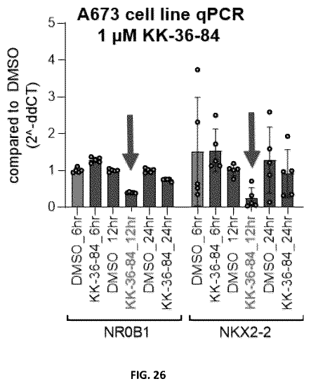

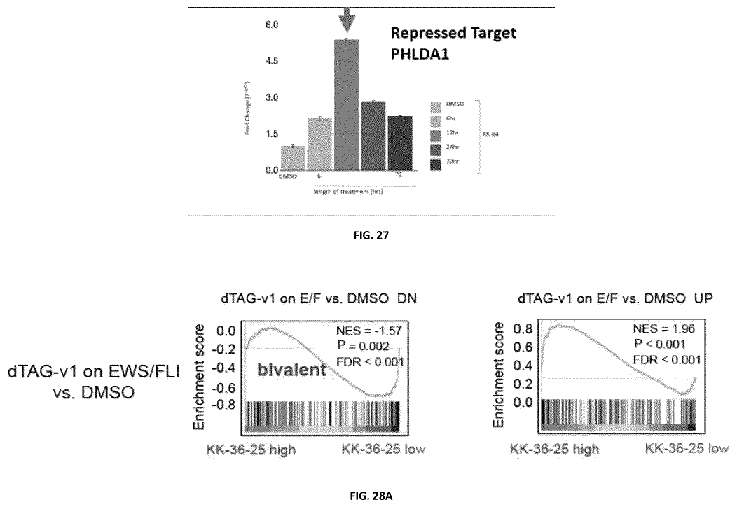

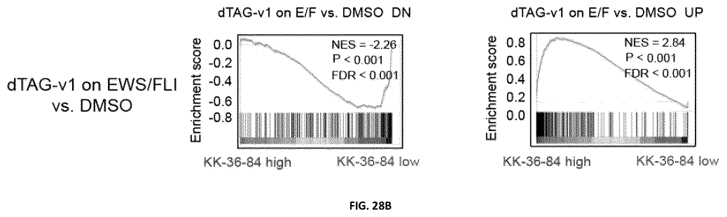

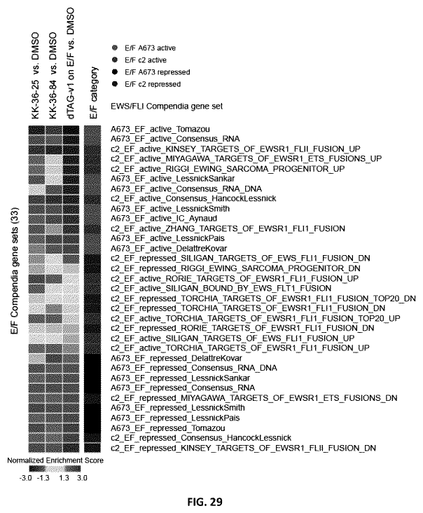

ERG Inhibitors Modulate Expression of Selected EWS-FLI1 Target Genes. FIG. 26 shows the effects of KK-36-84 expression of two genes (via qPCR) that are activated by EWS-FLI1 in the Ewings sarcoma cell line A673. FIG. 27 shows the effects of KK-36-84 on one gene (via qPCR) that is repressed by EWS-FLI1 in the Ewings sarcoma cell line A673. FIG. 28A shows GSEA analysis of RNASeq data after treatment with KK-36-25 compared to dTAG degradation of EWS-FLI1 (12 hours, EWS502 cell line). FIG. 28B shows GSEA analysis of RNASeq data after treatment with KK-36-84 compared to dTAG degradation of EWS-FLI1 (12 hours, EWS502 cell line). FIG. 29 shows the heatmap of normalized enrichment scores (ssGSEA) for treatment effects of KK-36-25, KK-36-84, and dTAG degradation of EWS-FLI1 on a compendia of EWS/FLI gene sets. In FIG. 29 the significance |Normalized Enrichment score (NES)|≥1.3; P-value 0.10; and FDR 0.10. The utility of targeting auto-inhibition to modulate ERG (FLI1) DNA binding and likely other members of the Eta family of transcription factors as well as transcription factors generally was demonstrated. To optimize potency, synthesized hetero-bivalent molecules combining an autoinhibition fragment with a fragment that reacts covalently with the protein. ERG/FLI inhibitors modulate the EWS-FLI1 gene expression program.

### REFERENCES

1. Sankar S, Lessnick S L. Promiscuous partnerships in Ewing's sarcoma. Cancer genetics 2011; 204(7):351-65 doi 10.1016/j.cancergen.2011.07.008.
2. Bailly R A, Bosselut R, Zucman J, Cormier F, Delattre 0, Roussel M, Thomas G, Ghysdael J. DNA-binding and transcriptional activation properties of the EWS-FLI-1 fusion protein resulting from the t(11;22) translocation in Ewing sarcoma. Molecular and cellular biology 1994; 14(5):3230-41.
3. Torchia E C, Jaishankar S, Baker S J. Ewing tumor fusion proteins block the differentiation of pluripotent marrow stromal cells. Cancer research 2003; 63(13):3464-8.
4. Gangwal K, Close D, Enriquez C A, Hill C P, Lessnick S L. Emergent Properties of EWS/FLI Regulation via GGAA Microsatellites in Ewing's Sarcoma. Genes & cancer 2010; 1(2):177-87 doi 10.1177/1947601910361495.
5. Gangwal K, Sankar S, Hollenhorst P C, Kinsey M, Haroldsen S C, Shah A A, Boucher K M, Watkins W S, Jorde L B, Graves B J, Lessnick S L. Microsatellites as EWS/FLI response elements in Ewing's sarcoma. Proceedings of the National Academy of Sciences of the United States of America 2008; 105(29):10149-54 doi 10.1073/pnas.0801073105.
6. Riggi N, Knoechel B, Gillespie S M, Rheinbay E, Boulay G, Suva M L, Rossetti N E, Boonseng W E, Oksuz O, Cook E B, Formey A, Patel A, Gymrek M, Thapar V, Deshpande V, Ting D T, Hornicek F J, Nielsen G P, Stamenkovic I, Aryee M J, Bernstein B E, Rivera M N. EWS-FLI1 utilizes divergent chromatin remodeling mechanisms to directly activate or repress enhancer elements in Ewing sarcoma. Cancer cell 2014; 26(5):668-81 doi 10.1016/j.ccell.2014.10.004.
7. Petrovics G, Liu A, Shaheduzzaman S, Furasato B, Sun C, Chen Y, Nau M, Ravindranath L, Dobi A, Srikantan V, Sesterhenn I A, McLeod D G, Vahey M, Moul J W, Srivastava S. Frequent overexpression of ETS-related gene-1 (ERG1) in prostate cancer transcriptome. Oncogene 2005; 24(23):3847-52.
8. Tomlins S A, Rhodes D R, Perner S, Dhanasekaran S M, Mehra R, Sun X W, Varambally S, Cao X, Tchinda J, Kuefer R, Lee C, Montie J E, Shah R B, Pienta K J, Rubin M A, Chinnaiyan A M. Recurrent fusion of TMPRSS2 and ETS transcription factor genes in prostate cancer. Science 2005; 310(5748):644-8 doi 10.1126/science.1117679.
9. Kong X T, Ida K, Ichikawa H, Shimizu K, Ohki M, Maseki N, Kaneko Y, Sako M, Kobayashi Y, Tojou A, Miura I, Kakuda H, Funabiki T, Horibe K, Hamaguchi H, Akiyama Y, Bessho F, Yanagisawa M, Hayashi Y. Consistent detection of TLS/FUS-ERG chimeric transcripts in acute myeloid leukemia with t(16;21)(p11;q22) and identification of a novel transcript. Blood 1997; 90(3):1192-9.
10. Marcucci G, Baldus C D, Ruppert A S, Radmacher M D, Mrozek K, Whitman S P, Kolitz J E, Edwards C G, Vardiman J W, Powell B L, Baer M R, Moore J O, Perrotti D, Caligiuri M A, Carroll A J, Larson R A, de la Chapelle A, Bloomfield C D. Overexpression of the ETS-related gene, ERG, predicts a worse outcome in acute myeloid leukemia with normal karyotype: a Cancer and Leukemia Group B study. J Clin Oncol 2005; 23(36):9234-42.
11. Baldus C D, Liyanarachchi S, Mrozek K, Auer H, Tanner S M, Guimond M, Ruppert A S, Mohamed N, Davuluri R V, Caligiuri M A, Bloomfield C D, de la Chapelle A. Acute myeloid leukemia with complex karyotypes and abnormal chromosome 21: Amplification discloses overexpression of APP, ETS2, and ERG genes. Proc Natl Acad Sci USA 2004; 101(11):3915-20.
12. Fotouhi N, Graves B. Small molecule inhibitors of p53/MDM2 interaction. Current topics in medicinal chemistry 2005; 5(2):159-65.
13. Miyazaki M, Naito H, Sugimoto Y, Kawato H, Okayama T, Shimizu H, Miyazaki M, Kitagawa M, Seki T, Fukutake S, Aonuma M, Soga T. Lead optimization of novel p53-MDM2 interaction inhibitors possessing dihydroimidazothiazole scaffold. Bioorganic & medicinal chemistry letters 2013; 23(3):728-32 doi 10.1016/j.bmcl.2012.11.091.
14. Zhang Z, Ding O, Liu J J, Zhang J, Jiang N, Chu X J, Bartkovitz D, Luk K C, Janson C, Tovar C, Filipovic Z M, Higgins B, Glenn K, Packman K, Vassilev L T, Graves B. Discovery of potent and selective spiroindolinone MDM2 inhibitor, R08994, for cancer therapy. Bioorganic & medicinal chemistry 2014; 22(15):4001-9 doi 10.1016/j.bmc.2014.05.072.
15. Zhao Y, Yu S, Sun W, Liu L, Lu J, McEachern D, Shargary S, Bernard D, Li X, Zhao T, Zou P, Sun D, Wang S. A potent small-molecule inhibitor of the MDM2-p53 interaction (MI-888) achieved complete and durable tumor regression in mice. Journal of medicinal chemistry 2013; 56(13):5553-61 doi 10.1021/jm4005708.
16. Pufall M A, Graves B J. Autoinhibitory domains: modular effectors of cellular regulation. Annual review of cell and developmental biology 2002; 18:421-62 doi 10.1146/annurev.cellbio.18.031502.133614.
17. Regan M C, Horanyi P S, Pryor E E, Jr., Sarver J L, Cafiso D S, Bushweller J H. Structural and dynamic studies of the transcription factor ERG reveal DNA binding is allosterically autoinhibited. Proceedings of the National Academy of Sciences of the United States of America 2013; 110(33):13374-9 doi 10.1073/pnas.1301726110.
18. Berger M, Dirksen U, Braeuninger A, Koehler G, Juergens H, Krumbholz M, Metzler M. Genomic EWS-FL11 fusion sequences in Ewing sarcoma resemble breakpoint characteristics of immature lymphoid malignancies. PloS one 2013;8(2):e56408 doi 10.1371/journal.pone.0056408.
19. Lin P P, Brody R I, Hamelin A C, Bradner J E, Healey J H, Ladanyi M. Differential transactivation by alternative EWS-FL11 fusion proteins correlates with clinical heterogeneity in Ewing's sarcoma. Cancer research 1999; 59(7):1428-32.
20. Giovannini M, Biegel J A, Serra M, Wang J Y, Wei Y H, Nycum L, Emanuel B S, Evans G A. EWS-erg and EWS-Fli1 fusion transcripts in Ewing's sarcoma and primitive neuroectodermal tumors with variant translocations. The Journal of clinical investigation 1994; 94(2):489-96 doi 10.1172/JC1117360.
21. Chen Y N, LaMarche M J, Chan H M, Fekkes P, Garcia-Fortanet J, Acker M G, Antonakos B, Chen C H, Chen Z, Cooke V G, Dobson J R, Deng Z, Fei F, Firestone B, Fodor M, Fridrich C, Gao H, Grunenfelder D, Hao H X, Jacob J, Ho S, Hsiao K, Kang Z B, Karki R, Kato M, Larrow J, La Bonte L R, Lenoir F, Liu G, Liu S, Majumdar D, Meyer M J, Palermo M, Perez L, Pu M, Price E, Quinn C, Shakya S, Shultz M D, Slisz J, Venkatesan K, Wang P, Warmuth M, Williams S, Yang G, Yuan J, Zhang J H, Zhu P, Ramsey T, Keen N J, Sellers W R, Stams T, Fortin P D. Allosteric inhibition of SHP2 phosphatase inhibits cancers driven by receptor tyrosine kinases. Nature 2016; 535(7610):148-52 doi 10.1038/nature18621.
22. Scott D E, Coyne A G, Hudson S A, Abell C. Fragment-based approaches in drug discovery and chemical biology. Biochemistry 2012; 51(25):4990-5003 doi 10.1021/bi3005126.
23. Illendula A, Pulikkan J A, Zong H, Grembecka J, Xue L, Sen S, Zhou Y, Boulton A, Kuntimaddi A, Gao Y, Rajewski R A, Guzman M L, Castilla L H, Bushweller J H. Chemical biology. A small-molecule inhibitor of the aberrant transcription factor CBFbeta-SMMHC delays leukemia in mice. Science 2015; 347(6223):779-84 doi 10.1126/science.aaa0314.
24. Illendula A, Gilmour J, Grembecka J, Tirumala V S, Boulton A, Kuntimaddi A, Schmidt C, Wang L, Pulikkan J A, Zong H, Parlak M, Kuscu C, Pickin A, Zhou Y, Gao Y, Mishra L, Adli M, Castilla L H, Rajewski R A, Janes K A, Guzman M L, Bonifer C, Bushweller J H. Small Molecule Inhibitor of CBFbeta-RUNX Binding for RUNX Transcription Factor Driven Cancers. EBioMedicine 2016; 8:117-31 doi 10.1016/j.ebiom.2016.04.032.
25. Choi A, Illendula A, Pulikkan J A, Roderick J E, Tesell J, Yu J, Hermance N, Zhu U, Castilla L H, Bushweller J H, Kelliher M A. RUNX1 is required for oncogenic Myb and Myc enhancer activity in T cell acute lymphoblastic leukemia. Blood 2017 doi 10.1182/blood-2017-03-775536.
26. Cooper D R, Boczek T, Grelewska K, Pinkowska M, Sikorska M, Zawadzki M, Derewenda Z. Protein crystallization by surface entropy reduction: optimization of the SER strategy. Acta crystallographica Section D, Biological crystallography 2007;63(Pt 5):636-45 doi 10.1107/S0907444907010931.
27. Putz M V, Duda-Seiman C, Duda-Seiman D, Putz A M, Alexandrescu I, Mernea M, Avram S. Chemical Structure-Biological Activity Models for Pharmacophores' 3D-Interactions. International journal of molecular sciences 2016;17(7) doi 10.3390/ijms17071087.
28. Melo-Filho C C, Braga R C, Andrade C H. 3D-QSAR approaches in drug design: perspectives to generate reliable CoMFA models. Current computer-aided drug design 2014;10(2):148-59.
29. Damale M G, Harke S N, Kalam Khan F A, Shinde D B, Sangshetti J N. Recent advances in multidimensional QSAR (4D-6D): a critical review. Mini reviews in medicinal chemistry 2014; 14(1):35-55.
30. Hollenhorst P C, McIntosh L P, Graves B J. Genomic and biochemical insights into the specificity of ETS transcription factors. Annual review of biochemistry 2011; 80:437-71 doi 10.1146/annurev.biochem.79.081507.103945.
31. Mammen M, Choi S K, Whitesides G M. Polyvalent interactions in biological systems: Implications for design and use of multivalent ligands and inhibitors. Angewandte Chemie-International Edition 1998; 37(20):2755-94.
32. Kiessling L L, Gestwicki J E, Strong L E. Synthetic multivalent ligands as probes of signal transduction. Angewandte Chemie-International Edition 2006; 45(15):2348-68.
33. Shuker S B, Hajduk P J, Meadows R P, Fesik S W. Discovering high-affinity ligands for proteins: SAR by NMR. Science 1996; 274(5292):1531-4.
34. Hajduk P J. SAR by NMR: putting the pieces together. Molecular interventions 2006; 6(5):266-72 doi 10.1124/mi.6.5.8.
35. Goldenberg D P. Computational simulation of the statistical properties of unfolded proteins. Journal of molecular biology 2003; 326(5):1615-33.
36. Dadlez M, KIm P S. Rapid formation of the native 14-38 disulfide bond in the early stages of BPTI folding. Biochemistry 1996; 35(50):16153-64 doi 10.1021/bi9616054.
37. Price-Carter M, Bulaj G, Goldenberg D P. Initial disulfide formation steps in the folding of an omega-conotoxin. Biochemistry 2002; 41(10):3507-19.
38. Winter G E, Buckley D L, Paulk J, Roberts J M, Souza A, Dhe-Paganon S, Bradner J E. DRUG DEVELOPMENT. Phthalimide conjugation as a strategy for in vivo target protein degradation. Science 2015; 348(6241):1376-81 doi 10.1126/science.aab1433.
39. Lai A C, Toure M, Hellerschmied D, Salami J, Jaime-Figueroa S, Ko E, Hines J, Crews C M. Modular PROTAC Design for the Degradation of Oncogenic BCR-ABL. Angewandte Chemie 2016; 55(2):807-10 doi 10.1002/anie.201507634.
40. Bondeson D P, Mares A, Smith I E, Ko E, Campos S, Miah A H, Mulholland K E, Routly N, Buckley D L, Gustafson J L, Zinn N, Grandi P, Shimamura S, Bergamini G, Faelth-Savitski M, Bantscheff M, Cox C, Gordon D A, Willard R R, Flanagan J J, Casillas L N, Votta B J, den Besten W, Famm K, Kruidenier L, Carter P S, Harling J D, Churcher I, Crews C M. Catalytic in vivo protein knockdown by small-molecule PROTACs. Nature chemical biology 2015; 11(8):611-7 doi 10.1038/nchembio.1858.
41. Gierisch M E, Pfistner F, Lopez-Garcia L A, Harder L, Schafer B W, Niggli F K. Proteasomal Degradation of the EWS-FL11 Fusion Protein Is Regulated by a Single Lysine Residue. The Journal of biological chemistry 2016; 291(52):26922-33 doi 10.1074/jbc.M116.752063.
42. Toure M, Crews C M. Small-Molecule PROTACS: New Approaches to Protein Degradation. Angewandte Chemie 2016; 55(6):1966-73 doi 10.1002/anie.201507978.
43. Ottis P, Crews C M. Proteolysis-Targeting Chimeras: Induced Protein Degradation as a Therapeutic Strategy. ACS chemical biology 2017; 12(4):892-8 doi 10.1021/acschembio.6b01068.
44. Collins I, Wang H, Caldwell J J, Chopra R. Chemical approaches to targeted protein degradation through modulation of the ubiquitin-proteasome pathway. The Biochemical journal 2017; 474(7):1127-47 doi 10.1042/BCJ20160762.
45. Gadd M S, Testa A, Lucas X, Chan K H, Chen W, Lamont D J, Zengerle M, Ciulli A. Structural basis of PROTAC cooperative recognition for selective protein degradation. Nature chemical biology 2017; 13(5):514-21 doi 10.1038/nchembio.2329.
46. Lu G, Middleton R E, Sun H, Naniong M, Ott C J, Mitsiades C S, Wong K K, Bradner J E, Kaelin W G, Jr. The myeloma drug lenalidomide promotes the cereblon-dependent destruction of Ikaros proteins. Science 2014; 343(6168):305-9 doi 10.1126/science.1244917.
47. Sufan R I, Ohh M. Role of the NEDD8 modification of Cul2 in the sequential activation of ECV complex. Neoplasia 2006; 8(11):956-63 doi 10.1593/neo.06520.
48. Tandefeldt et al., Endocrine Related Cancer 21(3): R143-152 (2014).
49. Carlton et al., Gynecol Oncol. 149(2):350-360 (2018).
50. Priest Chad et al., Patent PCT Int. Appl., 2012061698 10 May 2012.
51. Junghyun Chae et al., Org. Biomol. Chem., 2014, 12, 4747.
52. Peter Klein et al., Bioorg. Med. Chem. Lett. 2004, 14, 1455-1459.
53. Krysztof Jarowicki and Philip Kocienski J. Chem. Soc., Perkin Trans. 1, 1999, 1589-1615.

1. A compound of formula (I): 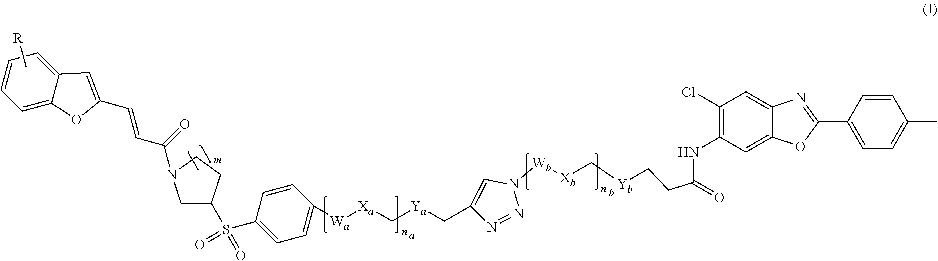
wherein:
R is, for each of the available binding sites, independently selected from the group consisting of H, F, Cl, Br, I, CH₃, OCH₃, CF₃, OCF₃, NO₂, NH₂, OH, N(CH₃)₂, CN, COCH₃, CONH₂, SO₂CH₃, and SO₂NH₂;
m is an integer from 1 to 4;
Wₐ, Xₐ, and Yₐ are for each occurrence independently O, CH₂, NH, cycloalkyl, benzyl, heterocycloalkyl, or heteroaryl, and one or more of Wₐ, Xₐ, and Yₐ are optionally not present, wherein the cycloalkyl, benzyl, heterocycloalkyl, and heteroaryl groups are independently unsubstituted or substituted, independently for each of the available binding sites, by H, F, Cl, Br, I, CH₃, OCH₃, CF₃, OCF₃, NO₂, NH₂, OH, N(CH₃)₂, CN, COCH₃, CONH₂, SO₂CH₃, or SO₂NH₂;
nₐ is an integer from 0 to 10;
W_{b}, X_{b}, and Y_{b} are for each occurrence independently O, CH₂, NH, cycloalkyl, benzyl, heterocycloalkyl, or heteroaryl, and one or more of W_{b}, X_{b}, and Y_{b} are optionally not present, wherein the cycloalkyl, benzyl, heterocycloalkyl, and heteroaryl groups are independently unsubstituted or substituted, independently for each of the available binding sites, by H, F, Cl, Br, I, CH₃, OCH₃, CF₃, OCF₃, NO₂, NH₂, OH, N(CH₃)₂, CN, COCH₃, CONH₂, SO₂CH₃, or SO₂NH₂; and
n_{b} is an integer from 0 to 10;
or a pharmaceutically acceptable salt thereof.

2. The compound of claim 1, wherein nₐ is an integer from 0 to 5.

3. The compound of claim 1, wherein nₐ is an integer from 0 to 3.

4. The compound of claim 1, wherein nₐ is 2.

5. The compound of claim 1, wherein nₐ is 1.

6. The compound of claim 1, wherein nₐ is 0.

7. The compound of claim 1, wherein Wₐ and Yₐ are each O and Xₐ is CH₂.

8. The compound of claim 1, wherein one of Wₐ and Xₐ is not present, the other of Wₐ and Xₐ is CH₂, and Yₐ is O.

9. The compound of claim 1, wherein n_{b} is an integer from 0 to 3.

10. The compound of claim 1, wherein n_{b} is 1.

11. The compound of claim 9, wherein one of W_{b} and X_{b} is not present, the other of W_{b} and X_{b} is CH₂, and Y_{b} is O.

12. The compound of claim 1, wherein the compound is selected from the group consisting of:

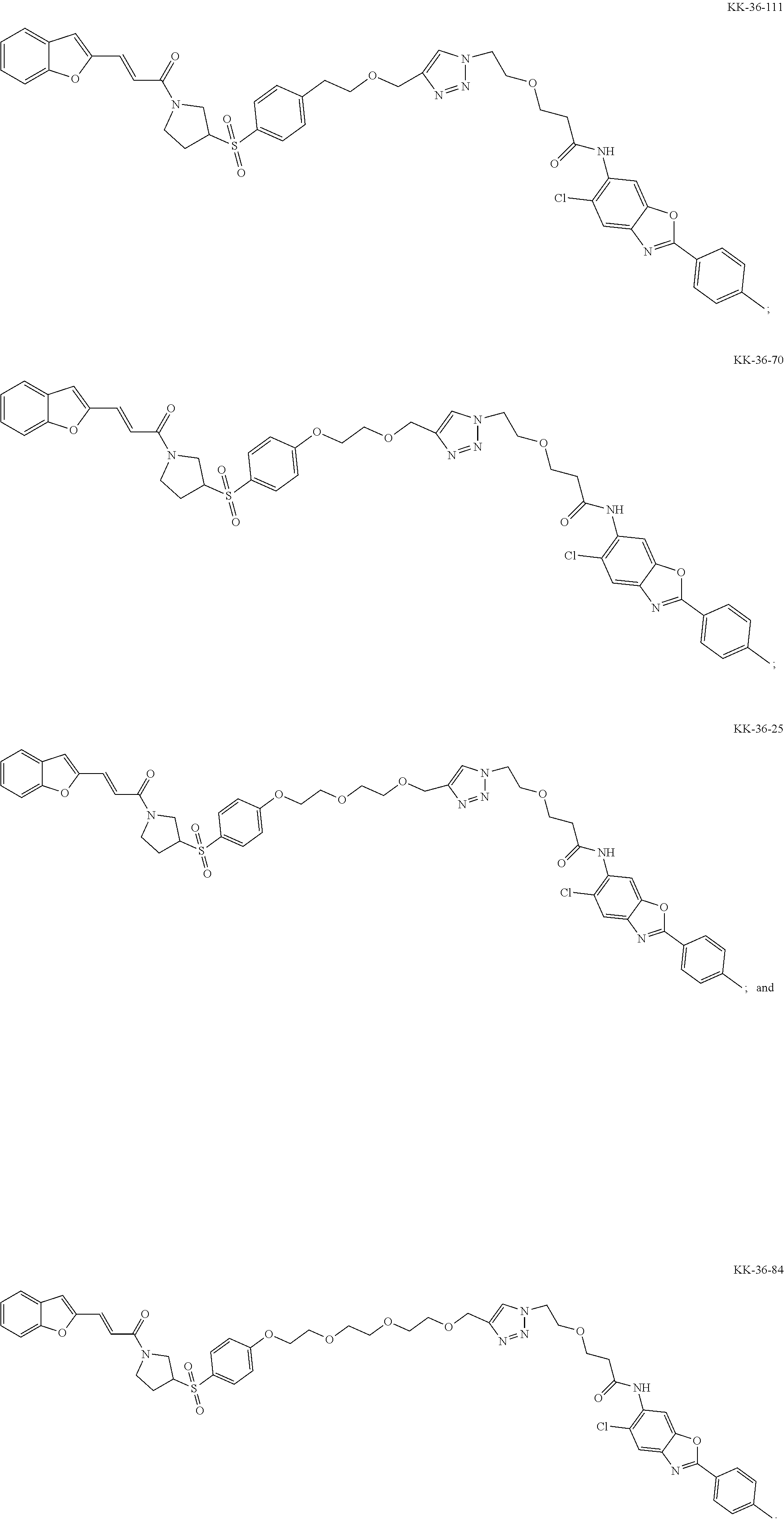 [SMILES: Cc1ccc(-c2nc3cc(Cl)c(NC(=O)CCOCCn4cc(COCCc5ccc(S(=O)(=O)C6CCN(C(=O)/C=C/c7cc8ccccc8o7)C6)cc5)nn4)cc3o2)cc1 ; Cc1ccc(-c2nc3cc(Cl)c(NC(=O)CCOCCn4cc(COCCOc5ccc(S(=O)(=O)C6CCN(C(=O)/C=C/c7cc8ccccc8o7)C6)cc5)nn4)cc3o2)cc1 ; Cc1ccc(-c2nc3cc(Cl)c(NC(=O)CCOCCn4cc(COCCOCCOc5ccc(S(=O)(=O)C6CCN(C(=O)/C=C/c7cc8ccccc8o7)C6)cc5)nn4)cc3o2)cc1 ; Cc1ccc(-c2nc3cc(Cl)c(NC(=O)CCOCCn4cc(COCCOCCOCCOc5ccc(S(=O)(=O)C6CCN(C(=O)/C=C/c7cc8ccccc8o7)C6)cc5)nn4)cc3o2)cc1]

13. A pharmaceutical composition comprising a compound of claim 1, and a pharmaceutically acceptable excipient.

14. A method of treating cancer, comprising administering to a subject in need thereof a therapeutically effective amount of a compound of claim 1.

15. A method of treating cancer, comprising administering to a subject in need thereof a therapeutically effective amount of a pharmaceutical composition of claim 13.

16. The method of claim 14, wherein the cancer is selected from the group consisting of Ewing sarcoma, leukemia, diffuse large B-cell lymphoma (DLBCL), and prostate cancer.

17. The method of claim 15, wherein the cancer is selected from the group consisting of Ewing sarcoma, leukemia, diffuse large B-cell lymphoma (DLBCL), and prostate cancer.

18. A process for preparing a compound of formula (I): 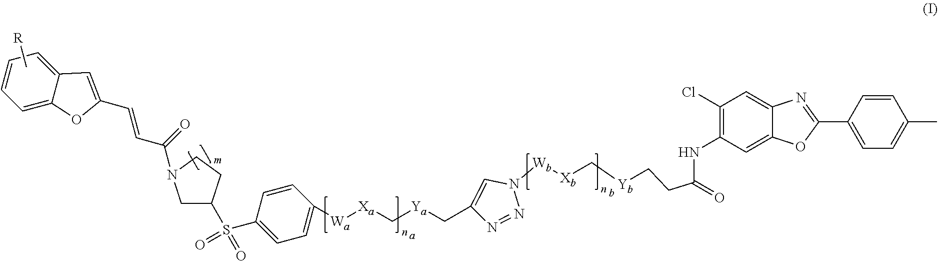
wherein in formula (I):
R is, for each of the available binding sites, independently selected from the group consisting of H, F, Cl, Br, I, CH₃, OCH₃, CF₃, OCF₃, NO₂, NH₂, OH, N(CH₃)₂, CN, COCH₃, CONH₂, SO₂CH₃, and SO₂NH₂;
m is an integer from 1 to 4;
Wₐ, Xₐ, and Yₐ are for each occurrence independently O, CH₂, NH, cycloalkyl, benzyl, heterocycloalkyl, or heteroaryl, and one or more of Wₐ, Xₐ, and Yₐ are optionally not present, wherein the cycloalkyl, benzyl, heterocycloalkyl, and heteroaryl groups are independently unsubstituted or substituted, independently for each of the available binding sites, by H, F, Cl, Br, I, CH₃, OCH₃, CF₃, OCF₃, NO₂, NH₂, OH, N(CH₃)₂, CN, COCH₃, CONH₂, SO₂CH₃, or SO₂NH₂;
nₐ is an integer from 0 to 10;
W_{b}, X_{b}, and Y_{b} are for each occurrence independently O, CH₂, NH, cycloalkyl, benzyl, heterocycloalkyl, or heteroaryl, and one or more of W_{b}, X_{b}, and Y_{b} are optionally not present, wherein the cycloalkyl, benzyl, heterocycloalkyl, and heteroaryl groups are independently unsubstituted or substituted, independently for each of the available binding sites, by H, F, Cl, Br, I, CH₃, OCH₃, CF₃, OCF₃, NO₂, NH₂, OH, N(CH₃)₂, CN, COCH₃, CONH₂, SO₂CH₃, or SO₂NH₂; and
n_{b} is an integer from 0 to 10;
or a pharmaceutically acceptable salt thereof;
comprising:
reacting a compound of formula (II) with a compound of formula (III) under conditions sufficient to form a compound of formula (IV): 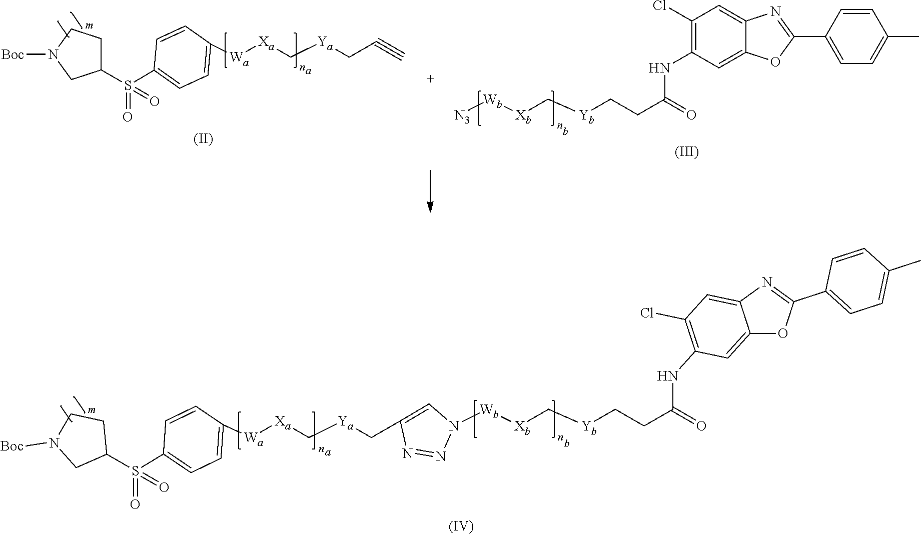
reacting the compound of formula (IV) under conditions sufficient to form a compound of formula (V): 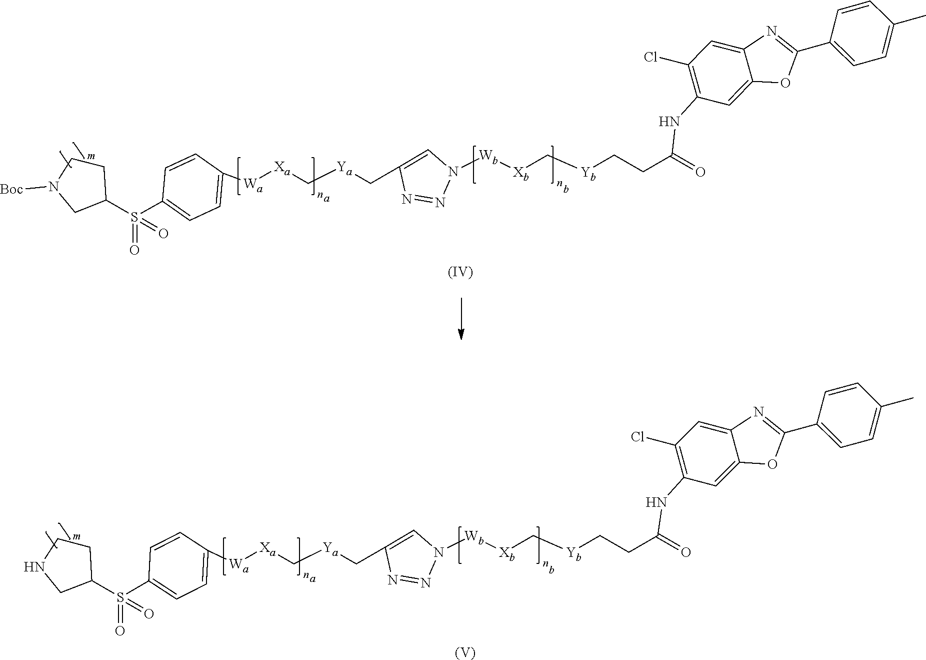
reacting the compound of formula (V) with a compound of formula (VI) under conditions sufficient to form the compound of formula (I): 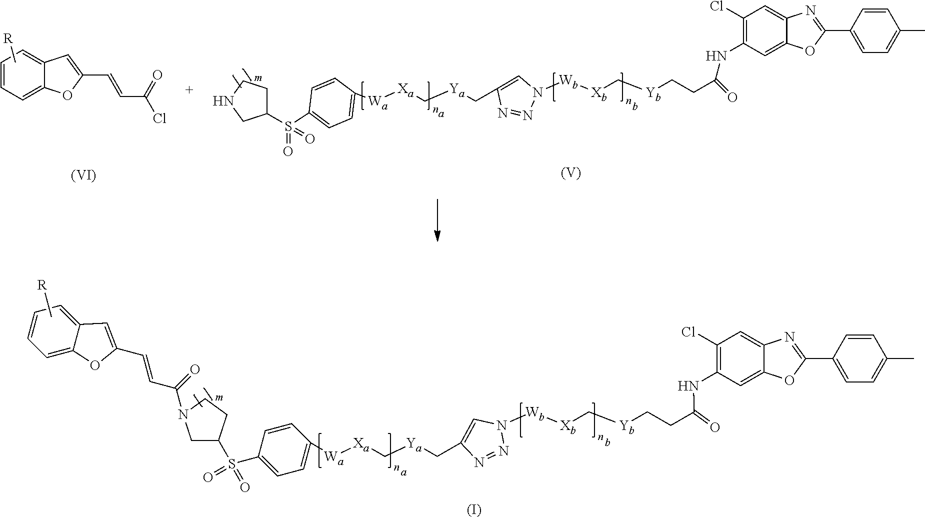
wherein R, m, Wₐ, Xₐ, Yₐ, nₐ, W_{b}, X_{b}, Y_{b}, and n_{b} in formulas (II), (Ill), (IV), (V), and (VI) are identical to how R, m, Wₐ, Xₐ, Yₐ, nₐ, W_{b}, X_{b}, Y_{b}, and n_{b} are defined in formula (I).

19. The process of claim 18, wherein the compound of formula (I) is selected from the group consisting of: 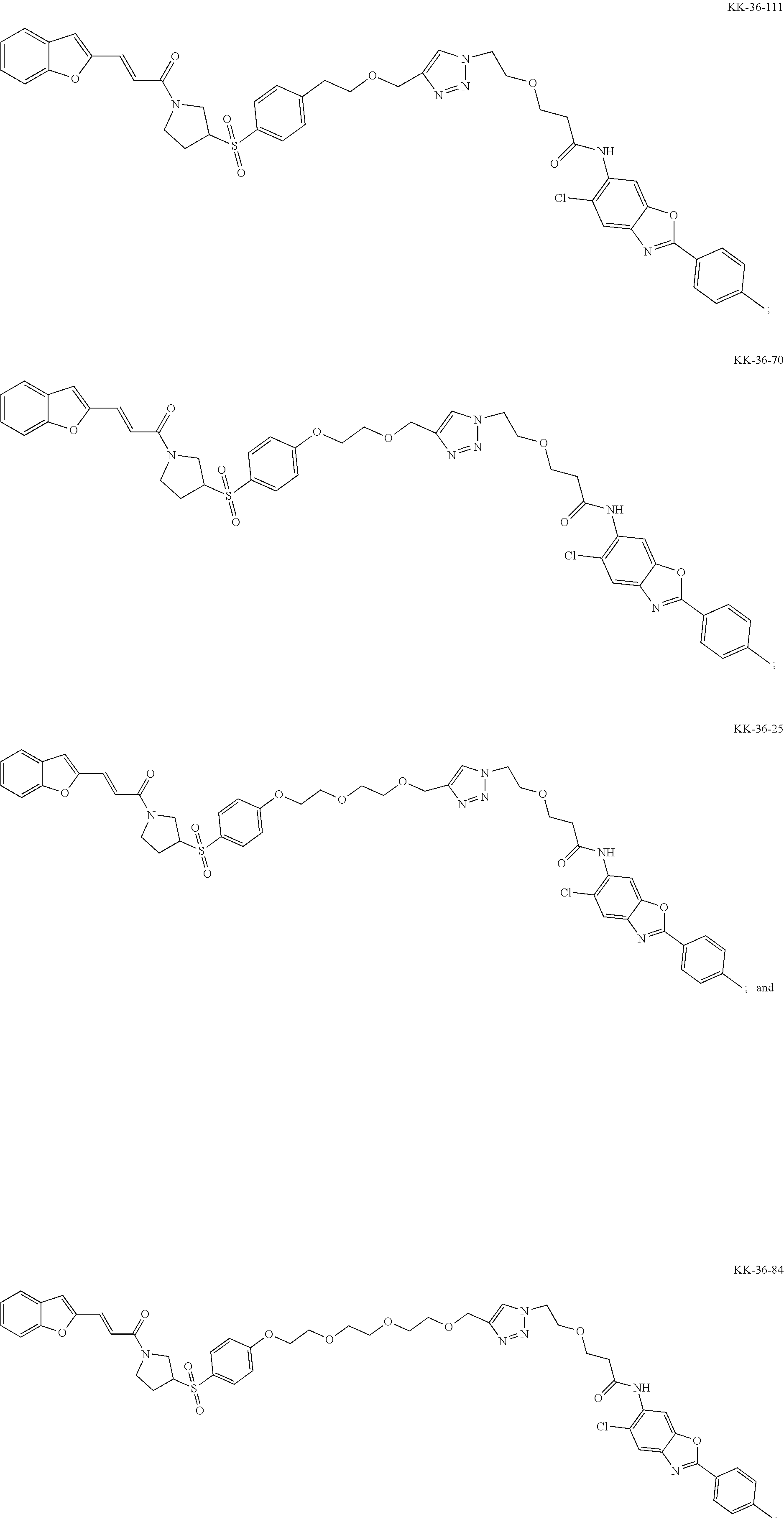 [SMILES: Cc1ccc(-c2nc3cc(Cl)c(NC(=O)CCOCCn4cc(COCCc5ccc(S(=O)(=O)C6CCN(C(=O)/C=C/c7cc8ccccc8o7)C6)cc5)nn4)cc3o2)cc1 ; Cc1ccc(-c2nc3cc(Cl)c(NC(=O)CCOCCn4cc(COCCOc5ccc(S(=O)(=O)C6CCN(C(=O)/C=C/c7cc8ccccc8o7)C6)cc5)nn4)cc3o2)cc1 ; Cc1ccc(-c2nc3cc(Cl)c(NC(=O)CCOCCn4cc(COCCOCCOc5ccc(S(=O)(=O)C6CCN(C(=O)/C=C/c7cc8ccccc8o7)C6)cc5)nn4)cc3o2)cc1 ; Cc1ccc(-c2nc3cc(Cl)c(NC(=O)CCOCCn4cc(COCCOCCOCCOc5ccc(S(=O)(=O)C6CCN(C(=O)/C=C/c7cc8ccccc8o7)C6)cc5)nn4)cc3o2)cc1]
